SEQUENCE LISTING (33 sequences)

SEQ ID NO: 1 - DNA, 800 bp, synthetic construct
  source 1..800: mol_type = other DNA
tgtgttggtgctggtgactcatggaatatgggcgcgtgtcttcctgaccaaatggtttagatggagcgtagaaaagttcgaagacctgcaaaacgtgcctaactgtcgctggatagtgatggaaaaggacgagacgacgcagcgttacgtccttcgcacgcatctgagcacgtggtcagagcttgaacagacaaaaagagaggaagaagtcaaaagagatgccgggagagagtttacattcaacagtacagtgcctctcacagacgacgaggtaagacaggtagccgacgcggaggcgcgagcgaaaaacgagcaggtgcaaaaatcgctaaaaataagcgattccgtcagactcactgcatctgggtcagagggaggctttaggacaggagccatctgtacagaggctacggagtgtggtggcgggttcatgggtggctcaagcggtctaaaagcaaaggtgcgcggccgtacgttattgtttgtgtgggtacgcgataaataaaaaatcagacaatcggctatgggggtgacataagcgatgagcaaactctatagcctcggcaaacctgggcgtgcacggatgtcgtcggagtgcaattttccagcggaccaaagttcaccaggaaaaaaatgacccaatatgggcggtgcccaatgatcacaccaacaattggtccacccctccccaatctctaatattcacaattcacctcactataaatacccctgtcctgctcccaaattcttttttccttcttccatcagctactagcttttatcttatttactttacgaaa

SEQ ID NO: 2 - DNA, 800 bp, synthetic construct
  source 1..800: mol_type = other DNA
tattttcacaattgcaccccagccagaccgatagccggccgcaatccgccacccacaaccgtctacctcccacagaaccccgtcacttccacccttttccaccagatcatatgtcccaacttgccaaattaaaaccgtgcgaattttcaaaataaactttggcaaagaggctgcaaaggaggggctggtgagggcgtctggaagtcgaccagacaccgggttggcggcgtatttgtgtcccaaaaaacagccccaattgccccaattgaccccaaattgacccagtagcgggcccaaccccggcgagagcccccttcaccccacatatcaaacctcccccggttcccacacttgccgttaagggcgtagggtactgcagtctggaatctacgcttgttcagactttgtactagtttcctaaaacatgcaatcggctgccccgcaacgggaaaaagaatgactttggcactcttcaccagagtggggtgtcccgctcgtgtgtgcaaataggctcccactggtcaccccggattttgcagaaaaacagcaagttccggggtgtctcactggtgtccgccaataagaggagcctaacgttcatgatcaaaatttaactgttctaacccctacttgacagcaatatataaacagaaggaagctgccctgtcttaaaccttttttttatcatcattattagcttactttcataattgcgactggttccaattgacaagcttttgattttaacgacttttaacgacaacttgagaagatcaaaaaacaactaattattcgaaacg

SEQ ID NO: 3 - DNA, 800 bp, synthetic construct
  source 1..800: mol_type = other DNA
cgaaatataccacattgccagtttatacagatggttaagggtgaaaatcaacgttacaccttgacgaccccattattacgatggcgtgaaggagatgaagaccgggtagaagaaataagaaaagcggtacagtttaggtccggagatctagggaaggaggccttagcttatattgtagctgctgagagagaggcagctgctggaagatctgaaggccctatcacgtatgatgatggtgatgaccattagagaacgcccagagattgatagccagttcttggacaaccgagtctctcggaaaacagcttctggatatcttccgctggcggcgcaacgacgaataatagtccctggaggtgacggaatatatatgtgtggagggtaaatctgacagggtgtagcaaaggtaatattttcctaaaacatgcaatcggctgccccgcaacgggaaaaagaatgactttggcactcttcaccagagtggggtgtcccgctcgtgtgtgcaaataggctctgctggagagcttcttctacggcccccttgcagcaatgctcttcccagcattacgttgcgggtaaaacggaggtcgtgtacccgacctagcagcccagggatggaaaagtcccggccgtcgctggctatataagcgggcggacgcatgtcatgagattattggaaaccaccagaatcgaatataaaacgcgaacacctttcccaattttggtttctcctgacccaaagactttaaatttaatttatttgtccctatttcaatcaattgaacaactatcaaaacaca

SEQ ID NO: 4 - DNA, 800 bp, synthetic construct
  source 1..800: mol_type = other DNA
cgaaatataccacattgccagtttatacagatggttaagggtgaaaatcaacgttacaccttgacgaccccattattacgatggcgtgaaggagatgaagaccgggtagaagaaataagaaaagcggtacagtttaggtccggagatctagggaaggaggccttagcttatattgtagctgctgagagagaggcagctgctggaagatctgaaggccctatcacgtatgatgatggtgatgaccattagagaacgcccagagattgatagccagttcttggacaaccgagtctctcggaaaacagcttctggatatcttccgctggcggcgcaacgacgaataatagtccctggaggtgacggaatatatatgtgtggagggtaaatctgacagggtgtagcaaaggtaatattttcctaaaacatgcaatcggctgccccgcaacgggaaaaagaatgactttggcactcttcaccagagtggggtgtcccgctcgtgtgtgcaaataggctcccactggtcaccccggattttgcagaaaaacagcaagttccggggtgtctcactggtgtccgccaataagaggagccggcaggcacggagtctacatcaactgttctaacccctacttgacagcaatatataaacagaaggaagctgccctgtcttaaaccttttttttatcatcattattagcttattcacaattcacctcactataaatacccctgtcctgctcccaaattcttttttccttcttccatcagctactagcttttatcttatttactttacgaaa

SEQ ID NO: 5 - DNA, 800 bp, synthetic construct
  source 1..800: mol_type = other DNA
cgaaatataccacattgccagtttatacagatggttaagggtgaaaatcaacgttacaccttgacgaccccattattacgatggcgtgaaggagatgaagaccgggtagaagaaataagaaaagcggtacagtttaggtccggagatctagggaaggaggccttagcttatattgtagctgctgagagagaggcagctgctggaagatctgaaggccctatcacgtatgatgatggtgatgaccattagagaacgcccagagattgatagccagttcttggacaaccgagtctctcggaaaacagcttctggatatcttccgctggcggcgcaacgacgaataatagtccctggaggtgacggaatatatatgtgtggagggtaaatctgacagggtgtagcaaaggtaatattttcctaaaacatgcaatcggctgccccgcaacgggaaaaagaatgactttggcactcttcaccagagtggggtgtcccgctcgtgtgtgcaaataggctcccactggtcaccccggattttgcagaaaaacagcaagttccggggtgtctcactggtgtccgccaataagaggagccggcaggcacggagtctacatcaactgttctaacccctacttgacagcaatatataaacagaaggaagctgccctgtcttaaaccttttttttatcatcattattataaaaggcgaacacctttcccaattttggtttctcctgacccaaagactttaaatttaatttatttgtccctatttcaatcaattgaacaactatcaaaacaca

SEQ ID NO: 6 - DNA, 800 bp, synthetic construct
  source 1..800: mol_type = other DNA
gttccggctccgatttctcggccaagtgccgactccacaagaccgaggaaggtaaactctctgttcacgtggggctgaagaaaggccagctcacggtggtggagttgcaatggaggcggcagctgaatagaccttgcaagtaaggggtacctgacgggaatgtgggaatatgggacacaaattggggggcaaacttgcaacatgcgtccagtgtaccccggataacaccattagtgtggctaatagcgaggatggggacttggagggatgggttcgcagggatggactcggagggatggacgcgaatggcgtggagggctcggatggcgcggagggttcggagatgggtccagggccaaaaatccggtttaaaaaggtggatatggtcgattgtagtggaagctcgtgatcctacatgctcacgaaacccatcatcatgcaatccacattaaaggaagggaaaaggatattgaacttttgactatttagtataaatgaaaactactttgtaagcttggacagaggaataatttctgattcgtgcttctgcttctactgacttgcaaaccagtaaacaagttactgcattaatcagtaataatctgcgtatagcgaaacttgtaaccctacttgacagcaatatataaacagaaggaagctgccctgtcttaaaccttttttttatcatcattattagcttactttcataattgcgactggttccaattgacaagcttttgattttaacgacttttaacgacaacttgagaagatcaaaaaacaactaattattcgaaacg

SEQ ID NO: 7 - DNA, 800 bp, synthetic construct
  source 1..800: mol_type = other DNA
aggtctttagtcagaggcaggaacagccgtcaagggggcataagactacggtcatccccatctgcctcttcgtccagccttgccaacagggagttcttcagagacatggaggctcaaaacgaaattattgacagcctagacatcaatagtcatacaacagaaagcgaccacccaactttggctgggggtagcgtataaacaatgcatactttgtacgttcaaaatacaatgcagtagatatatttatgcatattacatataatacatatcacataggaatggacagacaactataccagcatggatctcttgtatcggttcttttctcccgctctctcgcaataacaatgaacactgggtcaatcatagcctacacaggtgaacagagtagcgtttatacagggtttatacggtgattcctacggcaaaaatttttcatttctaaaaagaaaaagaaaaatttttctttccaacgctagaaggaaaagaaaaatctaattaaattgatttggtgattttctgagagttcccacacttgccgttaagggcgtagggtactgcagtctggaatctacgcttgttcagactttgtactagtttctttgtctggccatccgggtaacccatgccggacgctatataagctactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcaaa

SEQ ID NO: 8 - DNA, 800 bp, synthetic construct
  source 1..800: mol_type = other DNA
ttcgctcccacactacaccgtaataccacgtcactctcattgcaggttaccctgcccgtagtcgctcgatccacctcctccttctctcgtgtgtgcagcaaagaggcagagatggagcccgtatggtgaattaaaaccgtgcgaattttcaaaataaactttggcaaagaggctgcaaaggaggggggggtgagggcgtctggaagtcgaccagagaccgggttggcggcgtatttgtgtcccaaaaaacagccccaattgccccaagtccaccaccactggacacttgatttacaagtgcggtggtatggataagcgaaccattgaaaagtttgagaaggaagccgatgagcttggaaagggttctttcaagtacgcttgggttcttgacaagttgaaggctgagcgagagcgaggtatcaccattgatattgctctctggaagttcgagacccccaagtactacgttaccattattgatgctcccggtcaccgagatttcatcaagaatatgattaccggtacttccccacacttgccgttaagggcgtagggtactgcagtctggaatctacgcttgttcagactttgtactagtttctttgtctggccatccgggtaacccatgccggacgctatataagctactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcaaa

SEQ ID NO: 9 - DNA, 800 bp, synthetic construct
  source 1..800: mol_type = other DNA
aggtctttagtcagaggcaggaacagccgtcaagggggcataagactacggtcatccccatctgcctcttcgtccagccttgccaacagggagttcttcagagacatggaggctcaaaacgaaattattgacagcctagacatcaatagtcatacaacagaaagcgaccacccaactttggctgggggtagcgtataaacaatgcatactttgtacgttcaaaatacaatgcagtagatatatttatgcatattacatataatacatatcacataggaagcaacacttgatttacaagtgcggtggtatggataagcgaaccattgaaaagtttgagaaggaagccgatgagcttggaaagggttctttcaagtacgcttgggttcttgacaagttgaaggctgagcgagagcgaggtatcaccattgatattgctctctggaagttcgagacccccaagtactacgttaccattattgatgctcccggtcaccgagatttcatcaagaatatgattaccggtacttccccacacttgccgttaagggcgtagggtactgcagtctggaatctacgcttgttcagactttgtactagtttctttgtctggccatccgggtaacccatgccggacgctatataagctactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcaaa

SEQ ID NO: 10 - DNA, 800 bp, synthetic construct
  source 1..800: mol_type = other DNA
ggtattttcacaattgcaccccagccagaccgatagccggccgcaatccgccacccacaaccgtctacctcccacagaaccccgtcacttccacccttttccaccagatcatatgtcccaacttgccaaattaaaaccgtgcgaattttcaaaataaactttggcaaagaggctgcaaaggaggggggggtgagggcgtctggaagtcgaccagagaccgggttggcggcgtatttgtgtcccaaaaaacagccccaattgccccaattgaccccaaattgacccagtagcgggccagcgtatcccagcctagtgtatcccagcctagcctagcctaggccaaacctagccctctctagcctagcgcccagcagaaacaccgatgaagcaaagaagtaacagcaggaaagaaaaacaaacacaacaaaaaaaaacaagcagcatagcatcaacagaaatttctaaagagaaccaaattcaccccagaaacaaccgcacaaatacgacatccatccacctttcttttatcccacacttgccgttaagggcgtagggtactgcagtctggaatctacgcttgttcagactttgtactagtttctttgtctggccatccgggtaacccatgccggacgctatataagctactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcaaa

SEQ ID NO: 11 - DNA, 800 bp, synthetic construct
  source 1..800: mol_type = other DNA
aggtctttagtcagaggcaggaacagccgtcaagggggcataagactacggtcatccccatctgcctcttcgtccagccttgccaacagggagttcttcagagacatggaggctcaaaacgaaattattgacagcctagacatcaatagtcatacaacagaaagcgaccacccaactttggctgggggtagcgtataaacaatgcatactttgtacgttcaaaatacaatgcagtagatatatttatgcatattacatataatacatatcacataggaagcaacaggcgccatcccagcgtatcccagcctagtgtatcccagcctagcctagcctaggccaaacctagccctctctagcctagcgcccagcagaaacaccgatgaagcaaagaagtaacagcaggaaagaaaaacaaacacaacaaaaaaaaacaagcagcatagcatcaacagaaatttctaaagagaaccaaattcaccccagaaacaaccgcacaaatacgacatccatccacctttcttttatcccacacttgccgttaagggcgtagggtactgcagtctggaatctacgcttgttcagactttgtactagtttctttgtctggccatccgggtaacccatgccggacgctatataagctactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcaaa

SEQ ID NO: 12 - DNA, 800 bp, synthetic construct
  source 1..800: mol_type = other DNA
aggaaacctcgatgattctcccgttcttccatgggcgggtatcgcaaaatgaggaatttttcaaatttctctattgtcaagactgtttattatctaagaaatagcccaatccgaagctcagttttgaaaaaatcacttccgcgtttcttttttacagcccgatgaatatccaaatttggaatatggggtactctatcgggactgcagataatatgacaacaacgcagattacattttaggtaaggcataaattctacaggcacctgcgaggcaagcaatctactaatgtttatttttcgtccaacctaattgtggtttcaaagcgctatcaggtggggggtaagaggaatgtgagtggaaagcgaaaataactggcagctggggtcagatcccgtgatgccacctcttgtggtattttgaaacgcgtgttgcgattggccgcgagaacggaaaggaatatatttactgccgatcgcattttggcctcaaataaatcttgagcttttggacataggtctgtggacacatgtcatgttagtgtacttcaatcgccccctggatatagccccgacaataggccgtggcctcatttttttgccttccgcacatttccattgctcggtacccacaccttgcttctcctgcacttgccaaccttaatactggtttacattgaccaacatcttacaagcggggggcttgtctagggtatatataaacagtggctctcccaatcggttgccagtctcttttttcctttctttccccacagattcgaaatctaaactacacatcacaca

SEQ ID NO: 13 - DNA, 800 bp, synthetic construct
  source 1..800: mol_type = other DNA
ctttcatagaatatactatgatccgttagcaatttttgtcgtcaccttgttccaattccattgccatctgtttagctccagtttttcagttcactcactcatgatggaagtacaggaacgaaaagaaaaatgtttctgaaaaaaaaaagatgttggggggacaatttgtggaatccgcctggataagctcgagggaaaactggaagctaggaagtttgtgcacaaagaaagagtgaatacaagaagccaatagcccggcgtcctaaattgtacatttgtgtcacattatgaattacaggaagtcagaaaacaggcagcacatgtctcgcacatgcatgtccatcagacgagacattatgagacatgcacgcgtgtgagagacatagcaaaagtctctccagtacacacagaaagacacgttcacaatccaggcaccccacagagaaaaaaaaaagaagaagcccggaagctggcacgccatcatcaaccaccgctcggtttacacgcatcccaactgtctttttttcttttcctctcgtagggttgggaatccagtattgtgggctaatgaactgagtcacataatgtggttatgttccaatataggtaccacctttgttcaagatttagttttctaattgaatataaatacagaggttatttcaacctaattgagattaaggagagacttattttactatagtatatatttatttataattacttattgttactccaatccccaagtagattagatttaatcaatcacacacaaacatcaaaacaacaaattaacaaaa

SEQ ID NO: 14 - DNA, 800 bp, synthetic construct
  source 1..800: mol_type = other DNA
tttttttattttttgtccctccgggatggcaagagggacaaagaagaatcttcgttcttctttcttgttctcaacttcccagcttccgtgtgattaccctccgggacaacagaaaaactggcattcggtatccagggaatctgctgagaaggaaagaaaacgaaaaaaaaattgtacatttgtgtcacattatgaattacaggaagtcagaaaacaggcagcacatgtctcgcacatgcatgtccatcagacgagacattatgagacatgtcacattttttattttttaatttttcattacgcagcaaacatgcagcatccactaacttcagagattcctgtaagataagtggttcgttattttccggattccaattttggtggtgctccgaaaagtggaagctcgtgatcctacatgctcacgaaacccatcatcatgcaatccacattaaaggaagggaaaaggatatctgctggataattttcagaggcaacaaggaaaaattagatggcaaaaagtcgtctttcaaggaaaaatccccaccatctttcgagatcccctgtaacttattggcaactgaaagaatgaaaaggaggaaaatacaaaatatactagaactgaaaaaataaaagtataaatagagaagatatatgccaatacttcacaatgttcgaatctattcttcatttgcagctattgtaaaataataaaacatcaagaacaaacaagctcaacttgtcttttctaagaacaaagaataaacacaaaaacaaaaagtttttttaattttaatcaaaaa

SEQ ID NO: 15 - DNA, 800 bp, synthetic construct
  source 1..800: mol_type = other DNA
aaaacgaagattaagataaagttgggtaaaatccggggtaagaggcaagggggtagagaaaaaaaaaccggagtcattatatacgataccgtccagggtaagacagtgatttctagcttccacttttttcaatttctttttttcgttccaaatggcgtccacccgtacatccggaatctgacggcacaagagccgattagtggaagccacggttacgtgattgcggtttttttttcctacgtataacgctatgacggtagttgaagttcatcaaagtgttggacagacaactataccagcatggatctcttgtatcggttcttttctcccgctctctcgcaataacaatgaacactgggtcaatcatagcctacacaggtgaacagagtagcgtttatacagggtttatacggtgattcctacggcaaaaatttttcatttctaaaaaaaaaaagaaaaattttttgcttctgctggataattttcagaggcaacaaggaaaaattagatggcaaaaagtcgtctttcaaggaaaaatccccaccatctttcgagatcccctgtaacttattggcaactgaaagaatgaaaaggaggaaaatacaaaatatactagaactgaaaaaaaaaaagtataaatagagaagatatatgccaatacttcacaatgttcgaatctattcttcatttgcagctattgtaaaataataaaacatcaagaacaaacaagctcaacttgtcttttctaagaacaaagaataaacacaaaaacaaaaagtttttttaattttaatcaaaaa

SEQ ID NO: 16 - DNA, 800 bp, synthetic construct
  source 1..800: mol_type = other DNA
ttgctgggatcacccatacatcactctgttttgcctgaccttttccggtaatttgaaaacaaacccggtcacgaagcggagatccggcgataattaccgcagaaataaacccatacacgagaagtagaaccagccgcacatggccggagaaactcctgcgagaatttcgtaaactcgcgcgcattgcatctgtatttcctaatgcggcacttccaggcctcgagaactctgacatgcttttgacaggaatagacattttcagaatgttatccatatgcctttcgggtttttttccttccttttccatcatgaaaaatctctcgagaacgtttatccattgcttttttgttgtctttttccctcgttcacagaaagtctgaagaagctatagtagaactatgagctttttttgtttctgttttccttttttttctcgtaggaacaatttcgggcccctgcgtgttcttctgaggttcatcttttacatttgcttctgctggataattttcagaggcaacaaggaaaaattagatggcaaaaagtcgtctttcaaggaaaaatccccaccatctttcgagatcccctgtaacttattggcaactgaaagaatgaaaaggaggaaaatacaataatgtaaattttttttgaatataaaaggagattgaaaaattttttctagcagaaatgttttcaagttttaattgcaagtttcgtttgagtattcagttgtattttagttgatttgtagtttatttactagtattctcatagttctaactccaagagaagtaacattaaag

SEQ ID NO: 17 - DNA, 800 bp, synthetic construct
  source 1..800: mol_type = other DNA
aaaacgaagattaagataaagttgggtaaaatccggggtaagaggcaagggggtagagaaaaaaaaaccggagtcattatatacgataccgtccagggtaagacagtgatttctagcttccacttttttcaatttctttttttcgttccaaatggcgtccacccgtacatccggaatctgacggcacaagagccgattagtggaagccacggttacgtgattgcggtttttttttcctacgtatagcaacctgtattcggtcattgatgcatgcatgtgccgtgaagcgggacaaccagaaaagtcgtctataaatgccggcacgtgcgatcatcgtggcggggttttaagagtgcatatcacaaattgtcgcattaccgcggaaccgccagatattcattacttgacgcaaaagcgtttgaaataatgacgaaaaagaaggaagaaaaaaaaagaaaaataccgcttctaggcgggttatctactgatccgagcttcgctggcgggagaaggaaaaaaaaaattttttttttccttctgtttagtactggaacattgagaaggcgtgtcaattttgaataattagagtggtcaaaaaaattttttttgcttgggataccctttttcgataatgtaaattttttttgaatataaaaggagattgaaaaattttttctagcagaaatgttttcaagttttaattgcaagtttcgtttgagtattcagttgtattttagttgatttgtagtttatttactagtattctcatagttctaactccaagagaagtaacattaaag

SEQ ID NO: 18 - DNA, 800 bp, synthetic construct
  source 1..800: mol_type = other DNA
ggtcacaatgtttaaagctctgtcggtaaattgtgattgatccatgattagcaaaattgcagttgtagggaaagcaatttatacagcgtacagacatgagacgcttgtgatggtctggtgatctagagccgcacagaaacttctagcaatatctggtgtgtctgggtggttgctggtgtctggtctgcataacttggcctgaggatgcttatgtaatatggccttcctggtgcatggaagtggctggtggcaaaaaaaagtctgtgagcctcccaagcaatctactaatgtttatttttcgtccaacctaattgtggtttcaaagcgctatcaggtggggggtaagaggaatgtgagtggaaagcgaaaataactggcagctggggtcagatcccgtgatgccacctcttgtggtattttgaaacgcgtgttgcgattggccgcgagaacggaaaggaatatatttactgccgatcgcattttggcctcaaataaatcttgagcttttggacatagattatatgttctttcttggaagctctttcagctaatagtgaagtgtttcctactaaggatcgcctccaaacgttccaactacgggcggaggttgcaaagaaaacgggtctctcagcgaattgttctcatccatgagtgagtcctctccgtcctttcctcgcgcctgcctaatccatcgtccaacagagaggtcgctctccttatatatatagttgatccccctttttttctacccttgcaatttttttttgggaccaaagaaaagaaacaagactgatacaaaa

SEQ ID NO: 19 - DNA, 799 bp, synthetic construct
  source 1..799: mol_type = other DNA
gttagtagcaacctgaactcggtcattgatgcatgcatgtgccgtgaagcgggacaaccagaaaagtcgtctataaatgccggcacgtgcgatcatcgtggcggggttttaagagtgcatatcacaaattgtcgcattaccgcggaaccgccagatattcattacttgacgcaaaagcgtttgaaataatgacgaaaaagaaggaagaaaaaaaaagaaaaataccgcttctaggcgggttatctactgatccgagcttccactaggatagcacccaaacacctgcatatttggacgacctttaccaaatgagaaccctgtatttccttggccagagcctgagaaactgaaccctgatccagacattatcaataccttgggttattagtagtgtccgttatttttctgtttaggttacgattttgccagattttttgggaggagggaaacaaaagaaccagtgctacacgacctttaagtgccatcaggcatcctgttttctcgacctcatctcatcacatccgtcagtctgagctttcagttctcagttttcgattgactcttgccctgctgcgcgcacaccataccctggctccctctcatgcttctggcgttaccccggaatcgtacatccatgccgcgaatcccggacaggactcagacggatttcactatttgggcgggcttgctccgtctgtccaaggcaacatttatataagggtctgcatcgccggctcaattgaatcttttttcttcttctcttctctatattcattcttgaattaaacacacatcaaca

SEQ ID NO: 20 - DNA, 800 bp, synthetic construct
  source 1..800: mol_type = other DNA
aggtctttagtcagaggcaggaacagccgtcaagggggcataagactacggtcatccccatctgcctcttcgtccagccttgccaacagggagttcttcagagacatggaggctcaaaacgaaattattgacagcctagacatcaatagtcatacaacagaaagcgaccacccaactttggctgggggtagcgtataaacaatgcatactttgtacgttcaaaggtttggaacaacactaaactaccttgcggtactaccattgacactacacatccttaattccaatcctgtctggcctccttcaccttttaaccatcttgcccattccaactcgtgtcagattgcgtatcaagtgaaaaaaaaaaaattttaaatctttaacccaatcaggtaataactgtcgcctcttttatctgccgcactgcatgaggtgtccccttagtggaaaagaatactgagccaaccctggaggacagcaagggaaaaatacctacaacttgcttcataatggtcgtaaaacccggttcccacacttgccgttaagggcgtagggtactgcagtctggaatctacgcttgttcagactttgtactagtttctttgtctggccatccgggtaacccatgccggacgctatataagctactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcaaa

SEQ ID NO: 21 - DNA, 800 bp, synthetic construct
  source 1..800: mol_type = other DNA
cgaaatataccacattgccagtttatacagatggttaagggtgaaaatcaacgttacaccttgacgaccccattattacgatggcgtgaaggagatgaagaccgggtagaagaaataagaaaagcggtacagtttaggtccggagatctagggaaggaggccttagcttatattgtagctgctgagagagaggcagctgctggaagatctgaaggccctatcacgtatgatgatggtgatgaccattagagaacgcccagagattgatagccagttcttggacaaccgagtctctcggaaaacagcttctggatatcttccgctggcggcgcaacgacgaataatagtccctggaggtgacggaatatatatgtgtggagggtaaatctgacagggtgtagcaaaggtaatattttcctaaaacatgcaatcggctgccccgcaacgggaaaaagaatgactttggcactcttcaccagagtggggtgtcccgctcgtgtgtgcaaataggctcccactggtcaccccggattttgcagaaaaacagcaagttccggggtgtctctggaatctacgcttgttcagactttgtactagtttctttgtctggccatccgggtaacccatgccggacgctatataagctactgaaaatttttttgctttgtggttgggactttagccaagggtataaaacaccaccgtccccgaattacctttcctcttcttttctctctctccttgtcaactcacacccgaaatcgttaagcatttccttctgagtataagaatcattcaaa

SEQ ID NO: 22 - DNA, 800 bp, synthetic construct
  source 1..800: mol_type = other DNA
ggtggctcttcttgtttttcttactgaaaagagactggaaccgagctctcgaagaaggccggtaggtgcgttttcgcggcgaccgtcgcactatgtcccgcacgcgccgcttggtagactggccagcgtcgtcctggctgtgtgcatcgcttttgttgagctcgtgttggaatttcagtataagcgattccgtcagactcactgcatctgggtcagagggaggctttaggacaggagccatctgtacagaggctacggagtgtggtggcgggttcatgggtggctcaagcggtctaaaagcaaaggtgcgcggccgtacgttattgtttgcacccgtgtacataagcgtgaaatcaccacaaactgtgtgtatcaagtacatagtgacatttaaataatagcaagaacaacaataatagtagcgctactggaagcaccacgtaatagtggaaaagaactggaaaaaccgctataagatgcatactccggcggtcttacgcggagatacaagctctgctggagagcttcttctacggcccccttgcagcaatgctcttcccagcattacgttgcgggtaaaacggaggtcgtgtacccgacctagcagcccagggatggaaaagtcccggccgtcgctggctatataagcgggcggacgcatgtcatgagattattggaaaccaccagaatcgaatataaaacgcgaacacctttcccaattttggtttctcctgacccaaagactttaaatttaatttatttgtccctatttcaatcaattgaacaactatcaaaacaca

SEQ ID NO: 23 - DNA, 800 bp, synthetic construct
  source 1..800: mol_type = other DNA
ccaccgcagacgcccacctcgttagcgtccattgcgatcctctcggtacatttggttacattttgcgacaggttgaaatgaatcggccgacgctcggtagtcggaaagagccgggaccggccggcgagcataaaccggacgcagtaggatgtcctgcacgggtctttttgtggggtgtggagaaaggggtgcttggagatggaagccggtagaaccgggctgcttggggggatttggggccgctgggctccaaagaggggtaggcatttcgttggggttacgtaattgcggcatttgggtcctgcgcgcatgtcccattggtcagaattgcacccgtgtacataagcgtgaaatcaccacaaactgtgtgtatcaagtacatagtgacatttaaataatagcaagaacaacaataatagtagcgctactggaagcaccacgtaatagtggaaaagaactggaaaaaccgctataagatgcatactccggcggtcttacgcggagatacaagctctgctggagagcttcttctacggcccccttgcagcaatgctcttcccagcattacgttgcgggtaaaacggaggtcgtgtacccgacctagcagcccagggatggaaaagtcccggccgtcgctggctatataagcgggcggacgcatgtcatgagattattggaaaccaccagaatcgaatataaaacgcgaacacctttcccaattttggtttctcctgacccaaagactttaaatttaatttatttgtccctatttcaatcaattgaacaactatcaaaacaca

SEQ ID NO: 24 - DNA, 800 bp, synthetic construct
  source 1..800: mol_type = other DNA
tttttttattttttgtccctccgggatggcaagagggacaaagaagaatcttcgttcttctttcttgttctcaacttcccagcttccgtgtgattaccctccgggacaacagaaaaactggcattcggtatcccaggaatctgctgagaaggaaagaaaacgaaaaaaaaattgtacatttgtgtcacattatgaattacaggaagtcagaaaacaggcagcacatgtctcgcacatgcatgtccatcagacgagacattatgagacatgcacgcgtgtgagagacatagcaaaagtctctccagtacacacagaaagacacgttcacaatccaggcaccccacagagaaaaaataaagaagaagcccggaagctggcacgccatcatcaaccaccgctcggtttacacgcatcccaactgtcttttttttctggaatcctataatttcgggcccctgcgtgttcttctgaggttcatcttttacatttgcttctgctggataattttcagaggcaacaaggaaaaattagatggcaaaaagtcgtctttcaaggaaaaatccccaccatctttcgagatcccctgtaacttattggcaactgaaagaatgaaaaggaggaaacccctacttgacagcaatatataaacagaaggaagctgccctgtcttaaaccttttttttatcatcattattagcttactttcataattgcgactggttccaattgacaagcttttgattttaacgacttttaacgacaacttgagaagatcaaaaaacaactaattattcgaaacg

SEQ ID NO: 25 - DNA, 800 bp, synthetic construct
  source 1..800: mol_type = other DNA
cacatgtatagtacgttgcacatagtctacaatattcagcattcagcattcagtatacagcatatggctaaatgatcacaaatgtgattgatgatttgacacgactagaaaagagaacgaaaaagggaaattccatgtcacgtgcgttggcacgtgacatggaatatcgaagaaagaaaaaaaaaacgatctcgtcctagtggaagcccagagtctggtccccccggagtcttcccaaaacaagaagctgacacatgttgacacagttcatcaaagtgttggacagacaactataccagcatggatctcttgtatcggttcttttctcccgctctctcgcaataacaatgaacactgggtcaatcatagcctacacaggtgaacagagtagcgtttatacagggtttatacggtgattcctacggcaaaaatttttcatttctaaaaaaaaaaagaaaaattttttgcttctgctggataattttcagaggcaacaaggaaaaattagatggcaaaaagtcgtctttcaaggaaaaatccccaccatctttcgagatcccctgtaacttattggcaactgaaagaatgaaaaggaggaaaatacaaaatatactagaactgaaaaaaaaaaagtataaatagagaagatatatgccaatacttcacaatgttcgaatctattcttcatttgcagctattgtaaaataataaaacatcaagaacaaacaagctcaacttgtcttttctaagaacaaagaataaacacaaaaacaaaaagtttttttaattttaatcaaaaa

SEQ ID NO: 26 - DNA, 800 bp, synthetic construct
  source 1..800: mol_type = other DNA
aatgaggtctttagtcagaggcaggaacagccgtcaagggggcataagactacggtcatccccatctgcctcttcgtccagccttgccaacagggagttcttcagagacatggaggctcaaaacgaaattattgacagcctagacatcaatagtcatacaacagaaagcgaccacccaactttggctgataatagcgtataaacaatgcatactttgtacgttcaaaatacaatgcagtagatatatttatgcatattacatataatacatcaaagtgttggacagacaactataccagcatggatctcttgtatcggttcttttctcccgctctctcgcaataacaatgaacactgggtcaatcatagcctacacaggtgaacagagtagcgtttatacagggtttatacggtgattcctacggcaaaaatttttcatttctaaaaaaaaaaagaaaaattttttgcttctgctggataattttcagaggcaacaaggaaaaattagatggcaaaaagtcgtctttcaaggaaaaatccccaccatctttcgagatcccctgtaacttattggcaactgaaagaatgaaaaggaggaaaatacaaaatatactagaactgaaaaaaaaaaagtataaatagagaagatatatgccaatacttcacaatgttcgaatctattcttcatttgcagctattgtaaaataataaaacatcaagaacaaacaagctcaacttgtcttttctaagaacaaagaataaacacaaaaacaaaaagtttttttaattttaatcaaaaa

SEQ ID NO: 27 - DNA, 800 bp, synthetic construct
  source 1..800: mol_type = other DNA
ttcaggcggctacttgtatgtagcatccacgttcatgttttgtggatcagattaatggtatggatatgcacggttgaaatgaatcggccgacgctcggtagtcggaaagagccgggaccggccggcgagcataaaccggacgcagtaggatgtcctgcacgggtctttttgtggggtgtggagaggggggtgcttggagatggaagccggtagaaccgggctgcttggggggatttggggccgctgggctccaaagaggggtaggcatttcgttggggttacgtaatccgcaccgaagtgacaacatggacaatgtgacacgtagatacacgcaggaagcagctgtccacacacatttatcccgaaaaatagcccgcatcacatgcacgactcgtaaaaagaaaagagctgcgggccaaaggaccaataagtgccgaggaatgttaagccaaaagaacaacgacgatcgccagacaggtttagtgggagcagcagcagcagaggccgtgcaacggcaggagagagaggtctggcgaaaaggaggagacgggccatgcagtcggatttgccgtcacgggaccgcaacatgcttttcattgcagtccttcaactatccatctcacctcccccaatggcttttaactttcgaatgacgaaagcacccccctttgtacagatgactatttgggaccaatccaatagcgcaattgggtttgcatcatgtataaaaggagcaatcccccactagttataaagtcacaagtatctcagtatacccgtctaaccacacatttatcacc

SEQ ID NO: 28 - DNA, 800 bp, synthetic construct
  source 1..800: mol_type = other DNA
atcacgctacacttagctacagaataaagctcggtagcgccaacagcgttgacaaatagctcaagggcgtggagcacagggtttaggaggttttaatgggcgagaaggcgcgtagatgtagtcttcctcggtcccatcggtaatcacgtgtgtgccgatttgcaagacgaaaagccacgagaatggggcgggagaggggatggaagtccccgaacagcaaccagcccttgccctcgtggacataacctttcacttgccagaactctaagcgtcaccacggtatacaagcgcacgtagaagattgtggaagttgagctcgtgttggaatttcagtataagcgattccgtcagactcactgcatctgggtcagagggaggctttaggacaggagccatctgtacagaggctacggagtgtggtggcgggttcatgggtggctcaagcggtctaaaagcaaaggtgcgcggccgtacgttattgtttgtgtgggtacgcgataaataaaaaatcagacaatcggctatgggggtgacataagcgatgagcaaactctatagcctcggcaaacctgggcgtgcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaaca

SEQ ID NO: 29 - DNA, 800 bp, synthetic construct
  source 1..800: mol_type = other DNA
gtaggttgggttgggtgggagcacccctccacagagtagagtcaaacagcagcagcaacatgatagttgggggtgtgcgtgttaaaggaaaaaaaaagaagcttgggttatattcccgctctatttagaggttgcgggatagacgccgacggagggcaatggcgccatggaaccttgcggatatggggacgccgcggcggactgcgtccgaaccagctccagcagcgttttttccgggccattgagccgactgcgaccccgccaacgtgtcttggcccacgcactcatgtcatgttggtgcggcttgcacgggataatcagcagtctttttcggaatttgaaaatgtttggacggactacgaaaataaggagtttgatattaatgagagtaagtttttcaatgcaatggaagagaagaacgaagctgcagaagagcaaatagacgctttgaaacagcagatcttgaacatggatgcatacaccaaacagctctgggagcaagcaaagtcgtcctggaagctggctcctaaaatggaaatgcatgtggcaaagtaatggctcctagggtggacttcaaggatacactcacattgattggttgacaaggttacgtaaacttgtctccgaagacgaatttgtttacgaagatagcatttatttgtaggttattgattaggtaagatttattcaacgcaaactgggtgatgaatataaataagctcgggtattcctccgcaaccagaacttgaattatatacgtttcgattgatttccatcaaattataaattcaacaattgca

SEQ ID NO: 30 - DNA, 800 bp, synthetic construct
  source 1..800: mol_type = other DNA
cagacagtgacgagtcatacattctccgtataatatcgtgtatgtccagacgatagtcgtactcgtactcgttactgtaactactgtgcgagtactcgtgcatgtatcgtaggtattgtatgttcgagtacatacacatacgataccaaacactgcccactgttctgtcatgttagatcatggcggggccacgtgacttgcatgcaggtttggcattgaatattcagcgtggctactacaagtagtacatactgtatcaatacgattgtacatacggtactgggtttcgcaaacaacatggtaaaattacaaatgtgactatttttccactttttattttggtacactggcctttctttttgccatcagcgcaattcccgacacccgtgcaccgcgacacccaaaagttgtcattgaaattttctcgccgtttgagatcgcttttggaaaaaaagaaaaaaattacctggaagctcgtagaatccagggagccgaggaataaactgggggtgcacagccttacaccttgttatatatctcgagcacacgatcaaagtgctacaaaaccagtacgaccacatcagcgcacagcatatgcaaaatttggaccagtatctaaagaaaatggcccgctggagggaaattgtataaagatagacaaatcgactaaatttctaaaacacaaaataattttttatgatccacatggatggaagtgctcaagttattgcaatccttgggggatgagaacaagaaggacttgatgcgacgcagtgtcgaggtctttacttgtaccatct

SEQ ID NO: 31 - DNA, 800 bp, synthetic construct
  source 1..800: mol_type = other DNA
gtaggttgggttgggtgggagcacccctccacagagtagagtcaaacagcagcagcaacatgatagttgggggtgtgcgtgttaaaggaaaaaaaaagaagcttgggttatattcccgctctatttagaggttgcgggatagacgccgacggagggcaatggcgccatggaaccttgcggatatggggacgccgcggcggactgcgtccgaaccagctccagcagcgttttttccgggccattgagccgactgcgaccccgccaacgtgtcttggcgcaacattcgggagcagctggagcgtatgtgtgagcggatggggcttttggacgcaaaccagccggttttggaccacgaccgactgcttgtgaacgtgctgaagagcattgtggctggctttttcgtcaatgctgcgcagctgagccggtccggcgactcgtaccggtcgatgaaaaagaaccaggcggtgtggatgcatccgtcgtcggtgctgttcggcgtgaagccgccgccgaagctggtgatttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaaccttttttttatcatcattattagcttactttcataattgcgactggttccaattgacaagcttttgattttaacgacttttaacgacaacttgagaagatcaaaaaacaactaattattcgaaacg

SEQ ID NO: 32 - DNA, 800 bp, synthetic construct
  source 1..800: mol_type = other DNA
cagacagtgacgagtcatacattctccgtataatatcgtgtatgtccagacgatagtcgtactcgtactcgttactgtaactactgtgcgagtactcgtgcatgtatcgtaggtattgtatgttcgagtacatacacatacgataccaaacactgcccactgttctgtcatgttagatcatggcggggccacgtgacttgcatgcaggtttggcattgaatattcagcgtggctactacaagtagtacatactgtatcaatacgattgtacatacggtactcaccctttgctacagtatgtacatacaagggcgcaacagagccgagtttaatggtttgaaacctaggtggaagaggggcgggcgaggtatcgtactgtgggtgcgatagttcaccagtcacgctggtgggccattctcttagcacatttcccccctcccaagtcccctcaacccccaatgtaaccctcaacctcccacagctcagtagcacacgtgcaactagttagtaacaacccccctccgtccagcttctctttcacactgcttagagttcgacttctacttgagtgtggggagattgctcacaagtctggactgccacggatgactaaagtttgagcgtttaatccgccaaatgaccacactgtgcataaactcgattgccagcgaaatagagttgctttactaagcacaaagtctgttgagttggctgagacttggatttataaaacgctgcagcgtccctctccagaccttttctgcaacttgacattttcttgttaacgacaccatcacaca

SEQ ID NO: 33 - DNA, 800 bp, synthetic construct
  source 1..800: mol_type = other DNA
gggacaaagaagaatcttcgttcttctttcttgttctcaacttcccagcttccgtgtgattaccctccgggacaacagaaaaactggcattcggtatcccgggaatctgctgagaaggaaagaaaacgaaaaaaaaattgtacatttgtgtcacattatgaattacaggaagtcagaaaacaggggggacatgtctcgcacatgcatgtccatcagacgagacattatgagacatgcacgcgtgtgagacttagctacagaataaagctcggtagcgccaacagcgttgacaaatagctcaagggcgtggagcacagggtttaggaggttttaatgggcgagaaggcgcgtagatgtagtcttcctcggtcccatcggtaatcacgtgtgtgccgatttgcaagacgaaaagccacgagaataaaccgggagaggggatggaagtccccgaacagcaaccagcccttgccctcgtggacataacctttcacttgccagaactctaagcgtcaccacggtatacaagcgcacgtagaagatcctcgaatttgagacgagtcacggccccattcgcccgcgcaatggctcgccaacgcccggtcttttgcaccacatcaggttaccccaagccaaacctttgtgttaaaaagcttaacatattataccgaacgtaggtttgggcgggcttgctccgtctgtccaaggcaacatttatataagggtctgcatcgccggctcaattgaatcttttttcttcttctcttctctatattcattcttgaattaaacacacatcaaca

(19) United States
(12) Patent Application Publication
(10) Pub. No.: US 2025/0011782 A1
(43) Pub. Date: Jan. 9, 2025

(54) ARTIFICIAL YEAST PROMOTER REGIONS

(71) Applicant: Kaesler Nutrition GmbH, Cuxhaven (DE)

(72) Inventors: Heiko DIETZ, Wurster Nordseeküste (DE); Julia BAUER, Bremen (DE); Melissa KRACHT, Bremerhaven (DE)

(21) Appl. No.: 18/712,588

(22) PCT Filed: Nov. 23, 2022
(86) PCT No.: PCT/EP2022/082939

§ 371 Date: May 22, 2024

(30) Foreign Application Priority Data
Nov. 23, 2021 (EP) 21210056.4

(51) Int. Cl. C12N 15/113; C12N 1/16

(52) U.S. Cl. CPC C12N 15/113; C12N 1/165; C12N 2510/02

(57) ABSTRACT

The invention relates to an artificial yeast promoter region, comprising a TATA box at position −90±15 nucleotides (nt) and/or a TATA box at position −160±15 nt, an enhancer element at position −350±25 nucleotides, and a second enhancer element at position −600±50 nucleotides. The invention further relates to an expression construct comprising the artificial yeast promoter region, a yeast host cell, comprising the artificial yeast promoter region, and to methods of producing a protein of interest in a yeast host cell by employing the artificial yeast promoter region.

### FIELD

The invention relates to methods for optimizing expression of a protein of interest in a yeast host cell. Provided are artificial promoter sequences that can drive expression of a protein of interest to different levels in a variety of yeast host cells.

### BACKGROUND TO THE INVENTION

Production of proteins is a frequent activity in academic as well as industrial laboratories. However, the success rate of functional protein production varies substantially from case to case because there are many variables which potentially influence gene expression (Welch et al., 2009. PLoS ONE 4: e7002; Parret et al., 2016. Current Opinion Struct Biol 38: 155-162). Some of these variables can be easily manipulated by, for example, choice of a suitable expression vector, including a suitable promoter, and removal of premature start codons.

Yeasts such as Saccharomyces cerevisiae and Schizosaccharomyces pombe are renowned as production facilities for a protein of interest. Deletion mutants of every open reading frame (Scherens and Goffeau, 2004. Genome Biol 5: 229; Kim et al., 2010. Nat Biotechnol 28: 617-623) have resulted in a thorough understanding of the biological roles of many of the genes. The Saccharomyces Genome Database (SGD; available at yeastgenome.org/) provides information about each and every yeast gene, including the effects of over- and under-expression or deletion of a gene.

Furthermore, the long period of industrial usage have selected yeast species that are adapted to the process conditions and can tolerate the mechanical forces in a bioreactor, inhibitory substances and fermentation products. In addition, divers yeast products have obtained GRAS (generally recognized as safe) status by the FDA, while the European Food Safety Authority (EFSA) has provided a list of organisms, termed Qualified Presumption of Safety (QPS), which includes several yeast strains.

The knowledge of intracellular processes such as metabolism, secretion, transport, signaling and other pathways, and the availability of a large tool set for genetic engineering, have made yeasts the workhorse for a wide variety of applications.

The expression of a heterologous protein requires control over gene expression to optimize product formation. Transcriptional control, for example the strength of a promoter and possible feedback mechanisms, are critical points for yeast engineering. The introduction of a heterologous protein, combined with disruption of genes that may be disadvantageous for optimal expression of the heterologous protein, often requires multiple rounds of genetic engineering. Multiple use of the same or similar promoters often results in genetic instability of engineered yeast strains due to homologous recombination between stretches of identical sequences.

There is thus a need for a new generation of yeast promoter sequences that sufficiently differ from existing promoter sequences, allowing their use in combination with these existing promoter sequences. This new generation of yeast promoter sequences preferably drive expression of a protein at different levels, allowing expression of a gene at a requested level.

### BRIEF DESCRIPTION OF THE INVENTION

The invention described herein is based on numerous artificial yeast promoter regions, comprising different promoter elements such as enhancer elements and TATA boxes at varying positions, that were tested in different yeast strains. These tests resulted in the identification of a minimal number of promoter elements that provide tailored expression levels of a protein in the yeast strains tested.

The invention provides an artificial yeast promoter region, comprising a TATA box at position −90±15 nucleotides (nt), and/or a TATA box at position −160±15 nt, an enhancer element at position −350±25 nucleotides, and a second enhancer element at position −600±50 nucleotides, wherein said enhancer element at position −350±25 nucleotides is selected from a Asparagine-rich Zinc-Finger 1 (AZF1) binding element and a Multicopy suppressor of SNF1 mutation (MSN4) binding element, wherein said enhancer element at position −600±50 nucleotides is selected from a GlyColysis Regulation 1 (GCR1) binding element, a GCR2 binding element, and a PseudoHyphal Determinant 1 (PHD1) binding element, wherein all positions are relative to the start codon ATG, and wherein the sequences in between the indicated TATA boxes and enhancer elements lack known repressor elements. When present at the indicated positions, the enhancer elements are not positioned at their natural distance to the start codon. The enhancer element at position −350±25 nucleotides is preferably 5′-AAMRGMA or 5′-RVCCCCYR. The second enhancer element at position −600±50 nucleotides is preferably selected from 5′-WGGAWGMY, 5′-WGGAAGNM, and 5′-VMTGCRKV.

As is known to a person skilled in the art, the term and/or is used herein to indicate that a TATA box is present at position −90±15 nucleotides (nt), at position −160±15 nt, or at both positions −90±15 nucleotides (nt) and −160±15 nt.

Said artificial yeast promoter region is able to drive expression of a downstream protein in at least one of Kluyveromyces marxianus, Kluyveromyces lactis, Komagataella pastoris, Komagataella phaffii, Ogataea angusta, Yarrowia lipolytica, Schizosaccharomyces pombe, Rhodotorula mucilaginosa, Candida famata and Saccharomyces cerevisiae, preferably at least in one of K. phaffii, Y. lipolytica and S. cerevisiae. The nucleotide sequences of the promoter region are preferably identical to any one of SEQ ID NO:s 1-33, over a length of at least 600 nucleotides.

The invention further provides an expression construct comprising the artificial yeast promoter region of the invention, for expression of a protein of interest in a yeast. Said expression construct preferably comprises a nucleotide sequence encoding a protein of interest under control of the artificial yeast promoter region. Said nucleotide sequence encoding the protein of interest preferably is codon optimized for expression in a yeast host cell.

The invention further provides a yeast host cell, comprising the artificial yeast promoter region of the invention, or the expression construct according the invention.

The invention further provides a method of producing a protein of interest in a yeast host cell, comprising providing an expression construct according to the invention, transforming a yeast cell with the expression construct, expressing the protein of interest; and, optionally, at least partly purifying the protein of interest.

The invention further provides method of producing a protein of interest in a yeast host cell, comprising providing the yeast host cell comprising the expression construct according to the invention, expressing the protein of interest; and, optionally, at least partly purifying the protein of interest. In methods of the invention, the protein of interest is preferably tagged. In methods of the invention, the protein of interest may be co-expressed in the yeast cell with one or more of a protein disulfide isomerase, a flavin-linked sulfhydryl oxidase, and an oxidoreductase. In methods of the invention, the protein of interest may be part of a metabolic pathway, the method further comprising modulating the expression levels of one or more other enzymes in the metabolic pathway. Said metabolic pathway may be selected from the production of a biofuel, the breakdown of a carbohydrate, the production of a biopolyester, the production of a tocochromanol, and the production of an alkaloid.

### FIGURE LEGENDS

FIG. 1: Cultivation of K. phaffii in media containing three different carbon sources to express YFP as a reporter under control of different promoters with a high strength.

FIG. 2: Cultivation of K. phaffii in medium containing two different carbon sources to express YFP as a reporter under control of various promoters with a medium strength.

FIG. 3: Cultivation of Y. lipolytica to express GFP as a reporter under control of the different promoters as indicated with a high strength (A), or with a similar fluorescence (B), when compared to a reference promoter.

FIG. 4: Cultivation of S. cerevisiae to express YFP as a reporter under control of the different promoters with a high strength.

FIG. 5: Cultivation of S. cerevisiae to express YFP as a reporter under control of the different promoters with a medium strength.

FIG. 6: Cultivation of S. cerevisiae for production of geranylgeraniol under control of the promoter EPK14 compared to the reference promoter.

FIG. 7: Cultivation of K. phaffii in media containing three different carbon sources for expression of phytase AppA from E. coli under control of the promoters EPK2 and EPK3 compared to the reference promoter.

FIG. 8: Normalized specific activity of phytase with PDI co-expression compared to the AppA production strain, which did not express PDI.

### DETAILED DESCRIPTION OF THE INVENTION

### Definitions

The term “or”, as used herein is defined as “and/or” unless specified otherwise.

The term “a” or “an” as used herein is defined as “at least one” unless specified otherwise. When referring to a noun in the singular, the plural is meant to be included, unless it follows from the context that it should refer to the singular only.

The term “substantial(ly)”, as used herein, refers to the general character or function which is specified. When referring to a quantifiable feature, these term is in particular used to indicate that it is for at least 75%, more in particular at least 90%, even more in particular at least 95% of the indicated feature. For example, a “substantially pure” compound or protein refers to a purity of at least 95%, more preferably at least 96%, at least 97%, at least 98%, at least 99% or at least 100% pure compound or protein, as determined by standard analytical techniques known in the art.

The term “yeast”, as is used herein, refers to a eukaryotic, unicellular microorganism that is classified as a member of the kingdom fungus. A preferred yeast is a yeast of the Saccharomyces sensu stricto complex (Hittinger, 2013. Trends Genet 29: 309-317; Naseeb et al., 2017. Int J Syst Evol Microbiol 67: 2046-2052) such as the species Saccharomyces cerevisiae, a methylotrophic yeast such as Komagataella pastoris, Komagataella phaffii (both together formerly known as Pichia pastoris) and Ogataea angusta (formerly known as Hansenula polymorpha), a fission yeast such as Schizosaccharomyces pombe, a Kluyveromyces species such as K. lactis and K. marxianus, a Yarrowia species such as Y. lipolytica, and a Arxula species such as Arxula adeninivorans.

The term “gene,” as used herein, refers to a nucleic acid molecule comprising a protein-coding or RNA-coding sequence such as miRNA or siRNA, in an expressible form, operably linked to a regulatory sequence, including a promoter and optionally other regulatory sequences that are required to control expression of the coding sequence.

The term “heterologous gene”, as used herein, refers to any gene or coding sequence of a gene that does not naturally occur in the species wherein it is expressed.

The term “transformation”, as is used herein, refers to the introduction of a nucleotide sequence such as a plasmid into a cell. The term preferably refers to stable integration of a nucleotide sequence such as a plasmid into the genome of a cell.

The terms “encoding”, “coding for”, or “encoded by”, as used herein, refer to the information to guide translation of a nucleotide sequence of a nucleic acid molecule into a specified protein. As is known by a person skilled in the art, the information by which a protein is encoded is specified by codons, i.e. a trinucleotide sequence of DNA or RNA that corresponds to a specific amino acid. A nucleic acid molecule encoding a protein may comprise a non-translated sequence, e.g., an intron, interspersed with translated regions of the nucleic acid or may lack such an intervening non-translated sequence, e.g., as in complementary DNA (cDNA).

The term “complementary DNA (cDNA)”, as is used herein, refers to DNA synthesized from a single-stranded RNA such as a messenger RNA (mRNA) or microRNA (miRNA), in a reaction catalyzed by an enzyme termed reverse transcriptase.

The term “operably linked” refers to the association of two or more nucleic acid fragments on a single nucleic acid molecule so that the function of one is affected by the other. For example, a promoter region is operably linked to a coding sequence when the promoter region is capable of affecting the expression of said coding sequence. In other words, the coding sequence is under transcriptional control of the promoter region.

The term “express” or “expression”, as used herein, refers to the process of transcription or/or translation of a gene.

The term “promoter region”, as used herein, refers to a nucleotide sequence upstream or surrounding a transcription start site that controls binding of an RNA polymerase. A coding sequence or functional RNA is located downstream of a promoter.

The term “TATA box”, as is used herein, refers to an element in the promoter region that functions as initiating site for a transcription complex comprising TATA-binding protein. A TATA box consensus sequence comprises the nucleotide sequence 5′-TATA(A/T)A(A/T)(A/G), and thus includes the sequences 5′-TATAAAAA, 5′-TATAAAAG, 5′-TATAAATA, 5′-TATAAATG, 5′-TATATAAA, 5′-TATATAAG, 5′-TATATATA and 5′-TATATATG. However, small alterations from this consensus sequence, such as 1 or 2 alterations, may be tolerated.

The term “regulatory element”, as used herein, refers to a nucleotide sequence located upstream, interspersed with, or downstream of a promoter that influences transcription, RNA processing or stability, or translation of the associated coding sequence. A regulatory element may include an enhancer element, translational leader sequence (5′ untranslated regions (UTR), intron, trailer sequence (3′ UTR), and a polyadenylation sequence.

The term “enhancer element”, as used herein, refers to a specific nucleotide sequence that can stimulate promoter activity. In general, an enhancer functions independent of its exact position relative to a promoter.

The term “alteration”, as used herein, refers to a change in a nucleic acid sequence of a gene compared to the nucleic acid sequence of a non-altered gene. Said change preferably is a change that affects the functionality of the gene. Encompassed in the term alteration is deletion of one or more, including all, nucleotides from the gene, substitution of one or more nucleotides in the gene, and insertion of one or more nucleotides into the gene, or a combination thereof.

The term “substitution”, as used herein, refers at a nucleic acid level to a change of one or more nucleotides into one or more other nucleotides, whilst the total number of nucleotides remain the same. A “substitution” at the amino acid level is a change of one or more amino acid residues into one or more other amino acid residues, whilst the total number of amino acid residues remains the same.

The term “protein”, as is used herein, refers to a chain of amino acids arranged in a specific order determined by the coding sequence in a nucleic acid molecule encoding the protein.

The term “sequence identity”, as is used herein, refers to the percentage of identical residues determined by comparing two optimally aligned sequences over a comparison window. Said two sequences are preferably compared over the full length of the shortest of two sequences. The percentage is calculated by determining the number of positions at which an identical nucleic acid base or amino acid residue occurs in both sequences to yield the number of matched positions, dividing the number of matched positions by the total number of positions in the window of comparison, and multiplying the result by 100 to yield the percentage of sequence identity.

The term “purifying”, as used herein, refers to at least partly separating of a compound or protein of interest from its environment. Purifying encompasses separating a fluid comprising said compound or protein of interest from solids such as cells and debris to obtain a fluid comprising the compound or protein of interest and optionally one or more other substances. Purifying further encompasses removal of impurities and other substances until the compound or protein of interest is substantially pure.

### Artificial Yeast Promoter Regions

This invention provides an artificial yeast promoter region, comprising one or two TATA boxes and two enhancer elements. A first TATA box is present at position −90±15 nucleotides (nt) relative to the translation start codon ATG, while a second TATA box is present at position −160±15 nt. Said artificial promoter region is further characterized by the presence of two enhancer elements, a first element at position −350±25 nucleotides, and a second enhancer element at position −600±50 nucleotides.

The nucleotide sequences in between the indicated TATA boxes and enhancer elements were shuffled sequences from existing yeast promoter regions. Care was taken when designing these promoter regions to eliminate possible transcriptional repressor elements such as ACR1 (5′-ATGACGTCA; Vincent and Struhl, 1992. Mol Cell Biol 12: 5394-5405), Rox1 (WTTGWW; Pevny and Lovell-Badge, 1997. Curr Opin Genet Dev 7: 338-344), and Rgt 1 (5′-CGGANNA; Kim, 2009. Biochimie 91: 300-303).

Throughout this description, the single letter IUPAC code for nucleotides is used, wherein A represents adenine, G represents guanine, C represents cytosine, T represents thymine, Y represents a pyrimidine (C or T), R represents a purine (A or G), W represents weak (A or T), S represents strong (G or C), K represents keto (T or G), M represents amino (C or A), D represents A, G, T (not C), V represents A, C, G (not T), H represents A, C, T (not G), B represents C, G, T (not A) and X or N represents any base. A gap is denoted by a -.

Said first and second enhancer elements may be any enhancer element, provided that the first enhancer element differs from the second enhancer element, and that the first and second enhancer elements bind different transcription activating proteins. In addition, said first and second enhancer element preferably bind a transcription activating protein in substantially all yeasts, preferably at least in one or more of Saccharomyces cerevisiae, Komagataella pastoris, Komagataella phaffii, Ogataea angusta, Schizosaccharomyces pombe, Kluyveromyces lactis, Kluyveromyces marxianus, Yarrowia lipolytica, Rhodotorula mucilaginosa and Candida famata. Said first and second enhancer elements are not positioned at their natural position relative to the start codon ATG. For example, MSN4 encodes a Cys2His2 zinc finger protein that binds to a stress-responsive element termed STRE in the promoter region of several genes, having the consensus sequence 5′-RVCCCCYR, normally resides at around position.

An artificial yeast promoter region according to the invention may be provided by fixing two TATA boxes at positions −90±15 nucleotides (nt) and/or −160±15 nt, relative to the translation start codon ATG, a first enhancer element at position −350±25 nucleotides, and a second enhancer element at position −600±50 nucleotides. The sequences in between these elements may be obtained from common yeast promoter regions, but are preferably shuffled or scrambled so that they sufficiently differ from existing promoter sequences. A total length of a promoter region according to the invention is between 600 and 1200 nt, such as between 700 and 1000 nt, such as around 800 nt.

Said first and second enhancer element may provide a binding site for any known yeast transcription factor such as, for example, Sterile 12 (Ste12), Heme Activator Protein 1 (Hap1), Asparagine-rich Zinc-Finger 1 (AZF1; YOR113W), Multicopy suppressor of SNF1 mutation (MSN4), GlyColysis Regulation 1 (GCR1), GCR2 and PseudoHyphal Determinant 1 (PHD1; YKL043W). An overview of suitable transcription factors is provided in, for example, Struhl, 1993. Current Opinion Cell Biol 5: 513-520, and in Gordan et al., 2011. Genome Biol 12: R125.

A preferred artificial yeast promoter region according to the invention comprises a first enhancer element at position −350±25 nucleotides selected from an Asparagine-rich Zinc-Finger 1 (AZF1; YOR113W) binding element and a Multicopy suppressor of SNF1 mutation (MSN4) binding element.

AZF1 is a Cys2His2 zinc finger transcription factor that activates transcription of genes involved in carbon metabolism, energy production on glucose, cell wall organization and biogenesis on glycerol-lactate. AZF1 binds a consensus sequence 5′-AAMRGMA, preferably 5′-AAAAGAA.

MSN4 (YKL062W) is also a zinc finger protein that activates genes in response to several stresses, including heat shock, osmotic shock, oxidative stress, low pH, glucose starvation, sorbic acid and high ethanol concentrations. MSN4 binds to a consensus sequence 5′-CCCCT, preferably 5′-RVCCCCYR.

A preferred artificial yeast promoter region according to the invention comprises a second enhancer element at position −600±50 nucleotides is selected from a GCR1 binding element, a GCR2 binding element, and a PHD1 binding element.

GlyColysis Regulation 1 (GCR1; YPL075W) is a transcriptional activator that drives expression of glycolytic and ribosomal genes. GCR1 binds to a core 5′-GGAAG sequence, termed CT box, preferably 5′-WGGAWGMY).

Similar to GCR1, GCR2 (YNL199C) also is a transcriptional activator that drives expression of glycolytic and ribosomal genes. GCR2 interacts and functions with the DNA-binding protein GCR1. GCR2 binds to a core 5′-WGGAAGNM sequence.

PseudoHyphal Determinant 1 (PHD1; YKL043W) is a transcriptional activator that enhances pseudohyphal growth. PHD1 binds to a core 5′-VMTGCRKV sequence.

A preferred artificial yeast promoter region is able to drive expression of a downstream protein in several yeasts. Said yeasts preferably include at least one of Kluyveromyces marxianus, Kluyveromyces lactis, Komagataella pastoris, Komagataella phaffii, Ogataea angusta, Yarrowia lipolytica, Schizosaccharomyces pombe, Rhodotorula mucilaginosa and Candida famata. and Saccharomyces cerevisiae, preferably at least in one of K. phaffii, Y. lipolytica and S. cerevisiae, more preferably at least two of K. phaffii, Y. lipolytica and S. cerevisiae.

A preferred artificial yeast promoter region comprises a nucleotide sequence that is at least 60% identical to one or more of SEQ ID NO:s 1-33, preferably at least 80% identical to one or more of SEQ ID NO:s 1-33, over a length of at least 600 nucleotides. Said preferred artificial yeast promoter region comprises a nucleotide sequence that is at least 85% identical, at least 90% identical, at least 91% identical, at least 92% identical, at least 93% identical, at least 94% identical, at least 95% identical, at least 96% identical, at least 97% identical, at least 98% identical, at least 99% identical, at least 100% identical to one or more of SEQ ID NO:s 1-33 over a continuous stretch of at least 600 nucleotides, more preferred at least 650 nucleotides, more preferred at least 700 nucleotides, more preferred at least 750 nucleotides, more preferred over their full length to one or more of SEQ ID NO:s 1-33.

The invention further provides a method of producing an artificial yeast promoter region, comprising a TATA box at position −90±15 nucleotides (nt) and/or a TATA box at position −160±15 nt, an enhancer element at position −350±25 nucleotides, and a second enhancer element at position −600±50 nucleotides, wherein all positions are relative to the start codon ATG, wherein the enhancer elements are not positioned at their natural distance to the start codon, and wherein the sequences in between the indicated TATA boxes and enhancer elements lack known repressor elements.

Said first enhancer element at position −350±25 nucleotides preferably is selected from AZF1 (5′-AAMRGMA) and MSN4 (5′-RVCCCCYR).

Said second enhancer element at position −600±50 nucleotides preferably is selected from a GCR1 binding element, preferably 5′-WGGAWGMY, a GCR2 binding element, preferably 5′-WGGAAGNM, and a PHD1 binding element, preferably 5′-VMTGCRKV.

The sequences in between the indicated TATA boxes and enhancer elements may be selected from existing yeast promoter regions and preferably comprise shuffled sequences from existing yeast promoter regions. Known sequences that may bind to transcriptional repressor elements such as ACR1 (5′-ATGACGTCA; Vincent and Struhl, 1992. Mol Cell Biol 12: 5394-5405), Rox1 (WTTGWW; Pevny and Lovell-Badge, 1997. Curr Opin Genet Dev 7: 338-344), and Rgt 1 (5′-CGGANNA; Kim, 2009. Biochimie 91: 300-303), preferably are avoided.

The nucleotide sequences of an artificial yeast promoter region according to the invention preferably is adjusted to mimic the GC-content of a yeast host organism. For example, for optimal expression in S. cerevisiae (GC content of 38.3%), an artificial yeast promoter region may comprise a GC content of between 30-50%. Similarly, for optimal expression in Yarrowia lipolytica (GC content of 49%), an artificial yeast promoter region may comprise a GC content of between 40-60%.

### Expression Constructs and Cells

The invention further provides an expression construct comprising an artificial yeast promoter region of the invention, for expression of a protein of interest in a yeast. For this, the expression construct preferably comprises a nucleotide sequence encoding a protein of interest under control of the artificial yeast promoter region.

Said expression construct is either a construct that integrates into the yeast host genome, preferably at a pre-selected position in the yeast host genome, or an episomal expression vector. Expression constructs typically contain a yeast promoter and terminator sequences and a yeast selectable marker cassette, as is known to a person skilled in the art. Most yeast vectors can be propagated and amplified in E. coli to facilitate cloning. Hence, these constructs also contain an E. coli origin of replication and a selectable marker such as, for example, a beta-lactamase (Bla) gene for resistance to ampicillin. Furthermore, an yeast expression construct may include a secretion leader amino acid sequence that efficiently directs a protein of interest outside of the yeast host cell into the growth medium.

Said protein of interest is preferably selected from a pharmacologically active protein, an antibody or antibody fragment, a therapeutic protein, a peptide such as peptide hormone or an antimicrobial peptide, an enzyme such as a cellulase, a protease, a protease inhibitor, an aminopeptidase, an amylase, a carbohydrase, a carboxypeptidase, a catalase, a chitinase, a cutinase, a deoxyribonuclease, an esterase, an alpha-galactosidase, a beta-galactosidase, a glucoamylase, an alpha-glucosidase, a beta-glucosidase, an invertase, a laccase, a lipase, a mannanase, a mutanase, an oxidase, a pectinolytic enzyme, a peroxidase, a phospholipase, a phytase, a phosphatase, a polyphenoloxidase, a redox enzyme, a ribonuclease, a transglutaminase and a xylanase, or a combination thereof.

A nucleotide sequence encoding a protein of interest is preferably codon optimized or codon harmonized for expression in a yeast. The term “codon optimized”, as is used herein, refers to the selection of codons in a nucleotide sequence encoding a protein of interest for optimal expression of the protein of interest in a yeast host cell. A single amino acid may be encoded by more than one codons. For example, arginine and leucine are each encoded by a total of 6 codons. Codon optimization, i.e. the selection of a preferred codon for a specific yeast cell at every amino acid residue, plays a critical role, especially when proteins are expressed in a heterologous system. While codon optimization may play a role in achieving high gene expression levels, other factors such as secondary structure of the messenger RNA also need to be considered. Codon optimization is offered by commercial institutions, such as ThermoFisher Scientific, called Invitrogen GeneArt Gene Synthesis, GenScript, called GenSmart™ Codon Optimization, or GENEWIZ, called GENEWIZ's codon optimization tool.

The term “codon harmonized”, as is used herein, refers to the alignment of codon usage frequencies with those of the expression yeast, particularly within putative inter-domain segments where slower rates of translation may play a role in protein folding. Codon harmonization may be accomplished by algorithms such as provided by Angov et al., 2011 (Angov et al., 2011. In: Evans and Xu (eds) Heterologous Gene Expression in E. coli. Methods in Molecular Biology (Methods and Protocols), vol 705. Humana Press; Claassens et al., 2017. PLoS One 12: e0184355.

As is known to a person skilled in the art, gene products expressed from heterologous genes may be more prone to degradation caused by proteolytic activity than proteins that are naturally expressed in a yeast cell. Such proteins do not naturally occur in the environment wherein they are secreted and may thus be more prone to degradation. Care has to be taken not to include proteolytic cleavage sites in the protein of interest for a proteolytic enzyme from the host yeast cell. If necessary, one or more conserved amino acid alterations, in which an aliphatic amino acid residue is replaced for another aliphatic amino acid residue, a hydroxyl or sulfur/selenium-containing amino acid residue is replaced for another hydroxyl or sulfur/selenium-containing amino acid residue, an aromatic amino acid residue is replaced for another aromatic amino acid residue, a basic amino acid residue is replaced for another basic amino acid residue, and an acidic amino acid residue or amide thereof is replaced for another acidic amino acid residue or amide thereof, may be engineered in the protein of interest to efficiently stop degradation.

Said conserved amino acid replacements can be made for aliphatic amino acid residues glycine, alanine, valine, leucine, and isoleucine; hydroxyl or sulfur/selenium-containing amino acid residues serine, cysteine, selenocysteine, threonine and methionine, aromatic amino acid residues phenylalanine, tyrosine, and tryptophan, basic amino acid residues histidine, lysine, and arginine; and acidic amino acid residues and their amides aspartate, glutamate, asparagine, and glutamine.

The invention further provides a yeast host cell, comprising an artificial yeast promoter region or an expression construct according to the invention. Said yeast host cell preferably is selected from Candida hispaniensis, Kluyveromyces marxianus, Kluyveromyces lactis, Komagataella phaffii, Ogataea angusta, Yarrowia lipolytica, and Saccharomyces cerevisiae, more preferably K. phaffii, Y. lipolytica or S. cerevisiae.

### Production of Proteins

The invention further provides a method of producing a protein of interest in a yeast host cell, comprising providing an expression construct according to invention, wherein the expression of a protein of interest is controlled by an artificial yeast promoter region according to the invention, transforming a yeast cell with the expression construct; expressing the protein of interest; and, optionally, at least partly purifying the protein of interest.

Said transformation preferably involves the integration of the expression construct in the genome of the yeast host cell. This integration may be performed randomly, or at a chosen locus of the genome of the yeast host cell by homologous recombination, for example through the use of Cre/lox or a CRISPR-Cas recombination system. The robust and precise integration of an expression construct at a specific locus of the yeast genome allows stable, high expression levels of a protein of interest in a yeast host cell. Said high expression levels in general are reproducible for different proteins of interest.

The invention further provides a method of producing a protein of interest in a yeast host cell, comprising an expression construct wherein the expression of a protein of interest is controlled by an artificial yeast promoter region according to the invention, expressing the protein of interest; and, optionally at least partly purifying the protein of interest.

Following transformation, a yeast host cell expressing the protein of interest under control of an artificial yeast promoter region may be selected that has correctly integrated the expression that expresses the protein of interest. A selected yeast host cell may be grown, for example, in fed-batch or fermenter cultures, to very high cell densities reaching up to 150 g cell dry weight per litre.

Purification of a protein of interest may comprise a series of processes intended to isolate one or more proteins from a complex mixture, usually cells, tissues, and/or growth medium. Various purification strategies can be followed. For example, proteins can be separated based on size, for example in a method called size exclusion chromatography. Alternatively, or in addition, proteins can be purified based on charge, e.g. through ion exchange chromatography or free-flow-electrophoresis, or based on hydrophobicity (hydrophobic interaction chromatography). It is also possible to separate proteins based on molecular conformation, for example by affinity chromatography. Said purification may involve the use of a specific tag, for example at the N-terminus and/or C-terminus of the protein. After purification, the proteins may be concentrated. This can for example be carried out with lyophilization or ultrafiltration.

A protein of interest may be tagged in order to facilitate purification of the protein. A tag refers to the addition of a peptide to the amino (N) or carboxy (C) terminus of a protein. The addition of a tag allows to isolate or immobilize a protein. Commonly used tags include a poly-histidine tag such as a 6×(His) tag, a myc tag, a glutathione-S-transferase tag, a HiBiT tag (Promega, Madison, Wisconsin), a FLAG tag, a HA tag, or multimeric tags such as a triple FLAG tag

If required, a protein of interest may be co-expressed in a yeast host cell together with one or more proteins that may increase correct folding of a recombinantly expressed protein of interest in a yeast host cell. For this, one or more proteins such as a protein disulfide isomerase, for example protein disulfide isomerase PDI1 (YCL043C), ER protein Unnecessary for Growth (EUG1, YDR518W), or Multicopy suppressor of PDI1 deletion (MPD1; YOR288C); a flavin-linked sulfhydryl oxidase, for example Essential for Respiration and Viability (ERV2; YPR037C); and/or thiol oxidase such as ER Oxidation or Endoplasmic Reticulum Oxidoreductin (ERO1; YML130C), may be overexpression in a yeast host cell to assist in correct folding of a protein of interest. Genes encoding one or more of the indicated Saccharomyces genes, or a related gene from another organism such as a fungus or another yeast, may be co-expressed in the yeast host cell as an auxiliary protein to assist in the folding of the protein of interest.

Further auxiliary proteins or chaparones may include a spliced version of Homologous to Atf/Creb1 (HAC1; YFL031W), an ATPase such as KARyogamy 2 (KAR2; YJL034W), a glutathione peroxidase such as a phospholipid hydroperoxide glutathione peroxidase, for example glutathione peroxidase (GPX1; YKL026C), and proteins that may enhance translocation of the protein of interest out of the yeast host cell into the culture medium such as SECretory 1 (SEC1; YDR164C) and Suppressor of Loss of Ypt1 1 (SLY1; YDR189W).

### Metabolic Pathways

Tuning of expression is required when building synthetic pathways within organisms such as yeasts. In addition, tuning of individual protein expression levels is desired to optimize many industrial biotechnological processes. For this, the promoter region may be changed to fine tune the transcription level and consequently the amount of protein produced. Fine tuning of individual expression levels of proteins that function in a cascade may enhance the overall yield of the final product.

Such pathways include, for example, the production of a biofuel such as ethanol, 1-butanol and isopropanol, for which enzymes such as acetyl-CoA C-acyltransferase (EC 2.3.1.16; for example Peroxisomal Oxoacyl Thiolase 1 (POT1; YIL160C), acetoacetyl-CoA transferase, for example ERGosterol biosynthesis 10 (ERG10; YPL028W), and an acetoacetate decarboxylase play an important role (Nandy and Srivastava, 2018. Microbiol Res 207: 83-90), the breakdown of a carbohydrate such as cellulose, which includes the tuning of expression levels of several enzymes including, for example, endoglucanases and cellulase (Claes et al., 2020. Metabolic Engineering 59: 131-141), and production of a biopolyester such as polyhydroxyalkanoates (PHA), for which expression of, for example, β-ketothiolase, NADPH-linked acetoacetyl-CoA reductase and PHA synthase, needs to be carefully finetuned (Terentiev et al., 2004. Applied Microbiol Biotech 64: 376-381).

In addition, the production of a tocochromanol, which term refers to amphipathic molecules with a hydrophobic isoprenoid-derived hydrocarbon tail and a polar aromatic head obtained from the shikimate pathway, and which include α, β, γ, δ-tocopherols and tocotrienols. Tocotrienols are formed from two precursors, homogentisic acid (HGA) and geranylgeranyl pyrophosphate (GGPP). HGA is synthesized from 4-hydroxyphenylpyruvate (4-HPP) under catalysis of 4-hydroxyphenylpyruvate dioxygenase (HPPD), while GGPP is derived from 2C-Methyl-D-erythritol-4-phosphate (MEP) pathway. A recent report (Shen et al., 2020. Nat Commun 11: 5155) indicated that engineered yeast can produce tocotrienols at yield of up to 7.6 mg/g dry cell weight.

Furthermore, alkaloids have a wide range of pharmacological activities including antimalarial (e.g. quinine), antiasthma (e.g. ephedrine), anticancer (e.g. homoharringtonine), cholinomimetic (e.g. galantamine), vasodilatory (e.g. vincamine), antiarrhythmic (e.g. quinidine), analgesic (e.g. morphine), antibacterial (e.g. chelerythrine), and antihyperglycemic activities (e.g. piperine). Synthesis of alkaloids in yeast may provide a fast, cheap and easy production platform that will contribute to cure or at least palliate the suffering of millions of people. Recent reports have documented breakthrough achievements in the production of important intermediates in the synthesis of alkaloids in yeasts, such as members of the benzylisoquinoline alkaloid family, and the production of tropane alkaloids such as cocaine (Pyne et al., 2020. Nat Commun 11: 3337; Srinivasan and Smolke, 2020. Nature 585: 614-619).

### EXAMPLES

Unless otherwise stated, the present disclosure can be performed using standard procedures, as described, for example in Sambrook et al., (2014) Molecular Cloning: A Laboratory Manual (4 ed.), Cold Spring Harbor Laboratory Press, Cold Spring Harbor, N.Y., USA; Davis et al., (1995) Basic Methods in Molecular Biology, Elsevier Science Publishing, Inc., New York, USA; and Berger and Kimmel Eds, (1987). Methods in Enzymology: Guide to Molecular Cloning Techniques Vol. 152, S. L., Academic Press Inc., San Diego, USA, which are all incorporated by reference herein in their entireties.

### Example 1. Engineering of Synthetic Promoters by Rational Shuffling Method

Engineered promoter sequences were shuffled in the following way. One or two core sequences with a TATA-Box element or TATA-Box-like element at position −90 t 15 bp and/or −160±15 bp were linked together without changing the relation of the core promoter to the start codon ATG. TATA-Box-like element were changed to TATA-Box sequences. In addition, a first enhancer region was linked to the core element in a way that the binding sites of Saccharomyces cerevisiae transcription factor AZF1 (5′-AAMRGMA) or MSN4 (5′-RVCCCCYR) start a position −350 bp. A second enhancer element exhibit GCR1-bs (5′-WGGAWGMY), GCR2-bs (5′-WGGAAGNM), or PHD1-bs (5′-VMTGCRKV) at position −600 bp, wherein −bs denotes binding sequence. Thereby the elements were not set at their natural distance to the start codon. Additional point mutations were set to partly prevent specific enzyme cleavage sites within the promoter sequences.

The different promoter sequences were ordered as synthetic strings or gene product from Twist Bioscience (San Francisco, CA, USA) or Invitrogen GeneArt Gene Synthesis (Thermo Fisher Scientific Inc.) with appropriate sequence overhang for the dedicated vector system.

Surprisingly, the rational engineering concept was applicable on sequences from different host yeast species (Candida hispaniensis, Kluyveromyces marxianus, Kluyveromyces lactis, Komagataella phaffii, Ogataea angusta, Yarrowia lipolytica, S. cerevisiae) to drive expression in various yeast species (K. phaffii, Y. lipolytica and S. cerevisae).

### Example 2. Synthetic Promoters for YFP Expression in K. phaffii

The engineered promoter sequences and the reference promoter of Gen GAP1 (Debailleul et al., 2013. Microb Cell Fact 12: 129) were cloned via XmaI and EcoRI restriction sites into an expression vector (pKN95). This contains the yellow fluorescent protein YFP (Venus) gene in addition to a kanMX gene as a selection marker. Expression vectors and empty vector (pKN1; Nord et al., 1997. Nature Biotech 15: 772-777) were transformed into competent K. phaffii cells by electroporation. To characterize the YFP expression under control of different promoters, all strains were cultivated as biological triplicates in deepwell plates. The cultivation was performed at 28° C. and 70% humidity in a volume of 1.5 ml YPD medium (1% w/v yeast extract, 2% w/v peptone and 2% w/v glucose), supplemented with geneticin (G418; 500 μg/ml). The medium was inoculated with three colonies of each respective strain. After 90 h, the samples were diluted 1:20 with fresh medium. 200 μl of each sample was pipetted into optical bottom plates (Thermo Scientific 96 well, black) in technical triplicates. Fluorescence (excitation 485 nm/emission 520 nm) and OD600 were measured in plate reader (FLUOstar Omega, BMG Labtech). In further experiments, the carbon source was additional exchanged to provide glycerol and/or methanol instead of glucose.

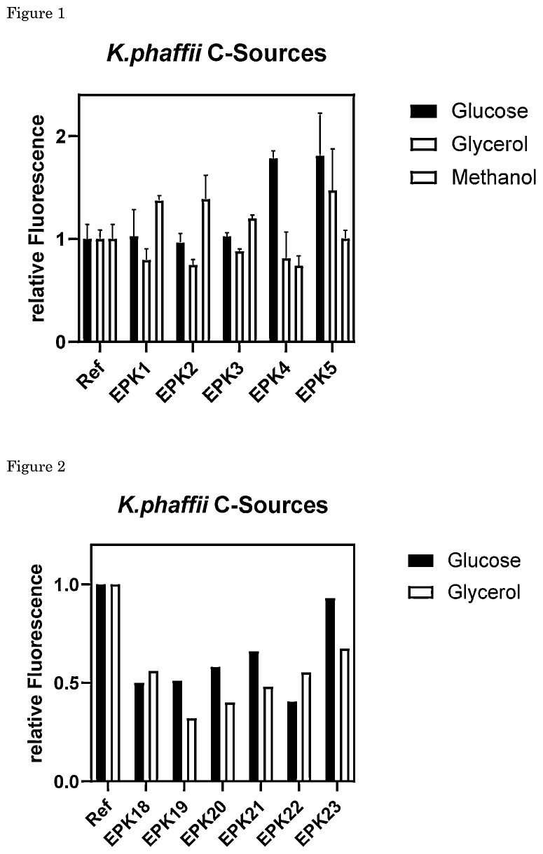

The measured fluorescence was normalized to OD600 and the reference promoter GAP1 was set to 1. In a set of around 45 sequences, the promoters EPK1 to 5 were designed at a later stage based on the good promoters. See FIG. 1.

As is shown in FIG. 2, the promoters EPK18 to 23 showed a lower fluorescence intensity relative to reference promoter GAP1, so these are interesting for other applications like the expression of auxiliary enzymes. By using promoters of different strengths, individual genes can be up or down regulated.

### Example 3. Synthetic Promoters for GFP Expression in Y. lipolytica

43 promoter sequences were cloned in front of a GFP cassette into an integrative vector. The integration locus IntC #2 from Y. lipolytica chromosome C was used for integration via CRISPR Cas9. Verified strains were cultivated in biological duplicates at 30° C. in YNB medium (20 g/L glucose and 0.67% yeast nitrogen base without amino acids). Additionally, transformants harbouring the TEF1 promoter were used as a positive control. After 24 h, cells were washed, diluted to an OD600 1.0 and 20 μl of each suspension was inoculated in 96 well plates with 180 μl YNB. The measurements were performed in the Synergy HT plate reader (BioTek, Vermont, USA). OD (600 nm) and GFP fluorescence (at 485 nm and emission at 528 nm) were measured.

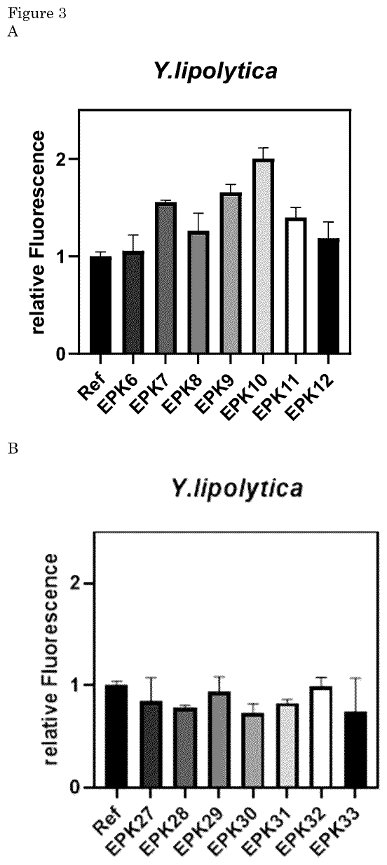

FIG. 3 shows the GFP fluorescence normalized to the OD600 after 24 h. The reference promoter TEF1 was set at 1. The strain with the construct EPK6 had a comparable fluorescence intensity than the TEF1 control. The strains bearing the promoters EPK7, EPK8, EPK9 EPK10, EPK11 and EPK12 showed a significant stronger fluorescence than strains with reference promoter (FIG. 3A). The strains bearing the promoters EPK27, EPK28, EPK29 EPK30, EPK31, EPK32 and EPK33 showed a similar fluorescence to strains with effective reference promoter (FIG. 3B).

### Example 4: Synthetic Promoters for YFP Expression in S. cerevisiae

The different promoter fragments and the reference promoter PGK1 were cloned in front of the YFP gene into a 2 expression vector containing an uracil cassette as a selective marker (Lee et al. 2015. ACS Synth. Biol. 2015, 4, 9, 975-986). These YFP expression vectors were chemically transformed into competent S. cerevisiae (CEN.PK2 1C) cells with the Li-Acetate, single stranded carrier DNA transformation protocol according to Gietz and Schiestl, 2007 (Gietz and Schiestl, 2007. Nat Protoc 2: 31-34). Interestingly, the differential YPF expression was already visible on the transformation plates. For further characterization of the YFP expression of the different promoters, all strains were cultivated in biological duplicates in 25 ml SCD-URA medium (Bruder et al. 2016. Microb Cell Fact 15: 127; supplemented with 2% w/v glucose) with a starting OD of 0.8 at 30° C. After 24 h, 20 μl samples were diluted with 180 μl fresh SCD-URA medium in a 96 well plate (Greiner 96 well, flat bottom, black) in technical triplicates. The fluorescence (497 nm excitation/540 nm emission) and the OD600 were measured in a plate reader (ClarioStar, BMG Labtech). The fluorescence intensity was normalized to the OD600 and PGK1 was set as a reference at 1.

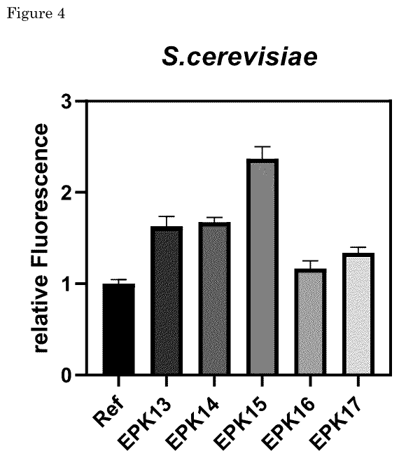

Our measurements revealed that 5 promoters of a set of 17 characterized variants showed a stronger fluorescence intensity than the positive control PGK1 (FIG. 4). The strain harbouring EPK15 showed the strongest fluorescence, followed by EPK13 and EPK14. The strains bearing EPK16 and EPK17 had a similar fluorescence intensity, which was still significantly higher than the reference promoter PGK1. Moreover, 3 medium strong promoters, EPK24, EPK25 and EPK26 were identified (FIG. 5).

### Example 5: Application of Synthetic Promoters for Metabolic Engineering of S. cerevisiae: Tocochromanol Production

a. Concept for Regulation of Gene Expression for Tocochromanol Production in Yeast

The group of tocochromanols comprises tocotrienols and tocopherols, commonly known as Vitamin E, and are naturally produced by photoautotrophic organisms. Due to rising demands in food, feed and cosmetic industry a sustainable production process for tocochromanols is required. The goal of these experiments is to obtain microorganisms as biotechnological production hosts.

Metabolic engineering of a microbial production hosts like yeast enables the synthesis of complex compounds from simple carbon sources like sugar.

Tocochromanols consist of a chromanol group, which is derived from homogentisic acid, and an isoprene chain that is derived from geranylgeranyl-diphosphate (GGPP). These two precursors can be converted into all Vitamin E isoforms by heterologous genes from plants and cyanobacteria.

For the production of homogentisic acid, a strongly expressed shikimic acid pathway is required: The endogenous genes ARO3 (UniProt #P14843) and ARO4 (P32449) need to be overexpressed under the control of strong synthetic promoters to direct the flux into the shikimic acid pathway. Moreover, these enzymes should be expressed in a feedback resistant version (Aro3K222L, in which a lysine (K) at position 222 is altered into a Leucine(L); and Aro4K229L, in which a lysine (K) at position 229 is altered into a Leucine (L)). Further, ARO1 (P08566), which encodes a pentafunctional enzyme, and ARO7 (P32178) have to be upregulated. Then, the flux needs to be directed into the tyrosine branch by overexpression of TYR1 (P20049). This effect can be enhanced if the genes (TRP2 (P00899), PHA2 (P32452)) of competing pathways for tryptophan and phenylalanine synthesis are downregulated with weak expressed promoters. Moreover, genes of degrading pathways need to be deleted or downregulated (ARO8 (P53090), ARO9 (P38840), ARO10 (Q06408) and PDC5 (P16467)). Thereby, the strains remain prototroph and do not require amino acid supplementation but enough precursor hydroxyphenylpyruvate (HPP) is synthesized. For the production of homogentisic acid from HPP the overexpression of the hydroxyphenylpyruvat dioxygenase (HPPD) (GenBank #VBB86065.1) is essential.

The second precursor of the isoprene chain is geranylgeranyl-diphosphate (GGPP). In yeast, GGPP is synthesized from the mevalonate pathway. Therefore, a truncated gene variant of the HMG1 (P12683) and the BTS1 (Q12051) gene, or a stronger heterologous variant like crtE from Xanthophyllomyces dendrorhous (Q1L6K3) are the key genes which need to be overexpressed by strong synthetic promoters. Moreover, it might be beneficial to regulate the expression of ERG20 (P08524) and IDI1 (P15496) to enhance the flux into GGPP synthesis.

Then, the two precursors homogentisic acid and GGPP will be prenylated by a heterologous homogentisat phytyl-transferase (HPT) and further cyclized be the tocopherol cyclase (TC) for tocotrienol production. To produce tocopherols, GGPP needs to be reduced by a heterologous GGPP reductase into phytyl-diphosphat (PDP). Moreover, two heterologous methyl-transferases, the γ tocopherol methyltransferase (γ-TMT) and the 2-methyl-6-phytylbenzoquinol methyltransferase (MPBQMT) need to be expressed under the control of synthetic promoters to synthesize α-, β- and γ-tocopherols and -tocotrienols from δ-tocopherol and δ-tocotrienol, respectively.

b. Example for the Regulation of the Mevalonate Pathway with Synthetic Promoters

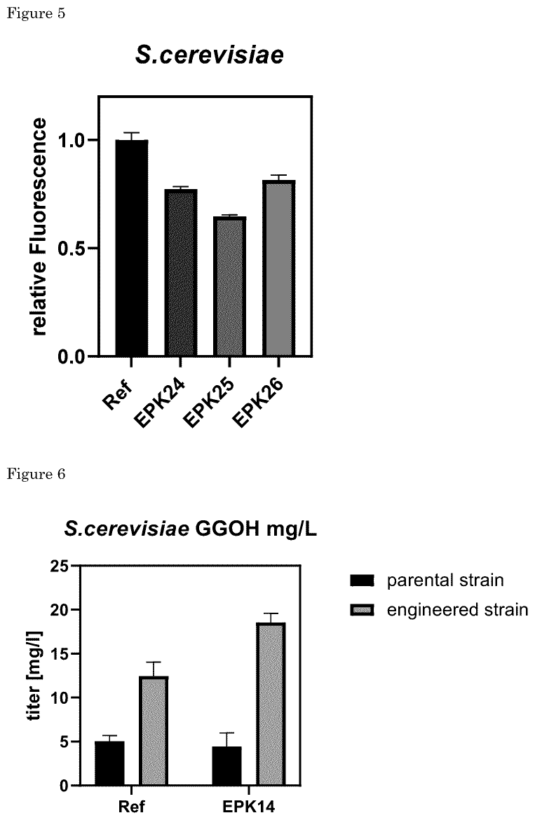

The synthetic promoter EPK14 was used in a project of metabolic engineering of S. cerevisiae for enhanced geranylgeraniol (GGOH) production. Two different production strains (JBY6 and JBY12) were generated from the modified CEN.PK2-1C strain (Entian and Kötter, 2007. Methods Microbiol 36: 629-666). The enzyme geranyl-geranyl diphosphate synthase (GGPPS) which is encoded by BTS1 was shown to be crucial for GGOH production from GGPP. Therefore, the two strains JBY6 (leu2Δ::pPGK1-BTS1-tADH1) and JBY12 (hoΔ::EPK14-BTS1-tADH1) were created by integration of a BTS1 overexpression cassette with either the reference promoter PGK1 (JBY6) or the synthetic promoter EPK14 (JBY12). The strains were verified on selective agar plates and by PCR methods. Since BTS1 is still limiting the GGOH production, the GGPPS was additionally overexpressed on 2 plasmids. All strains were cultivated for 144 h in 50 ml selective synthetic minimal medium+2% glucose in biological duplicates in shake flasks (30° C., 180 rpm). 2 ml samples of each flask was taken and the cell pellet was harvested. For cell disruption 500 μl methanol was added and the cells were shaken for 10 min, 60° C. at 1400 rpm. Then, the organic phase was harvested, evaporated overnight and resolved in ethyl acetate. The analysis was performed by gas chromatography with the Perkin Elmer Clarus 680 (Perkin Elmer) using the Elite 200 column (Perkin Elmer) (30 m, 0.25 mm ID, 0.25 mdf) with FID and helium as carrier gas. FIG. 6 shows the GGOH titers in mg/L after 144 h. The strains JBY6, harbouring the PGK1 promoter and JBY12, bearing the synthetic promoter EPK14 were able to produce similar amounts of GGOH (˜5 mg/L). Moreover, if BTS1 is additionally expressed on 2 plasmids the strain JBY12 is producing 18.55 mg/L whilst JBY6 did only produce 12.52 mg/L. This shows, exemplary for EPK14, that the synthetic promoters are applicable for gene expression in S. cerevisiae.

In metabolic engineering, many different promoters with specific properties are required because several genes need to be individually regulated. This method reveals a complete set of sequences to highly upregulate important genes and downregulate genes of contrary pathways.

More specifically, one of the weak promoters can be used to downregulate the competing ergosterol pathway. Either, farnesyl-diphosphate can be converted into the desired geranylgeranyl-diphosphate or into a precursor of ergosterol by ERG9 (P29704). The strong promoter EPK14 already leads to higher expression of BTS1, but the effect of upregulation can be enhanced by using one of the weak promoters so weaken the ERG9 expression and channel the flux from farnesyl-diphosphate into geranylgeranyl-diphosphate production.

c. Application of Synthetic Promoters for Metabolic Engineering of S. cerevisiae: Tocotrienol Production

The following example shows the applicability of the synthetic promoter EPK15 for heterologous gene expression in S. cerevisiae. For 8-tocotrienol production a yeast strain was constructed which is producing the two intermediates: homogentisic acid and GGPP. Moreover, two heterologous enzymes, the prenylase HPT (P73726) and the cyclase VTE1 (829413) are needed to form 8-tocotrienol. Both heterologous enzymes were integrated into the leu2 locus. The synthetic promoter EPK15 was used in front of the heterologous HPT gene of Synechocystis spec. and a truncated version of the VTE1 (deletion of the first 47 amino acids) gene was expressed under the control of PGK1 (JBY20). This strain was cultivated in biological triplicates in 50 ml YPD medium+15 ml dodecane overlay at 30° C. and 180 rpm. After 144 h, 1 ml of the dodecane phase was harvested and measured in the HPLC Bio-LC (Dionex) using the Agilent Zorbax SB-C8 column (4.6×150 mm, 3.5 μm). Buffer A (ddH2O+0.1% formic acid) and buffer B (acetonitrile+0.1% formic acid) were run in a gradient up to 100% buffer B for 40 min and delta tocotrienol was measured using UV detection at 300 nm. Furthermore, δ-tocotrienol was identified by mass determination in the LC-MS ([M+H]+ 397.3101). Using this strain, delta-tocotrienol was produced by S. cerevisiae, which gives another example for the usage of synthetic promoters for metabolic engineering in yeast.

### Example 6: Synthetic Promoters for Expression of AppA from Escherichia coli in K. phaffii

The promoters EPK2 and EPK3, and the reference promoter GAP1 were cloned into an expression vector via the restriction sites XmaI and EcoRI. This vector contains the AppA (P07102, amino acids 23-432) gene from E. coli in addition to a kanMX gene as a selection marker. These AppA expression vectors as well as the empty vector were transformed into electro-competent K. phaffii cells. After expression of the enzyme, it is secreted into the culture supernatant so that phytase activity can be measured in the medium. The activity of phytase can therefore be used as a direct signal of promoter strength.

To characterize the AppA expression under the different promoters, all strains were cultivated in biological triplicates in deep well plates. Cultivation was performed at 28° C. and 70% humidity in a volume of 1.5 ml YPD medium added with geneticin (G418; 500 μg/ml). The medium was inoculated with three colonies of each respective strain. After 90 h, 100 μl of each samples were diluted with fresh medium. 200 μl of each sample was pipetted into 96-well plates (StarLab, 96 well plates, round, flat bottom) in technical triplicates. The OD600 were measured in a plate reader (FLUOstar Omega, BMG Labtech).

Subsequently, the cells were centrifuged at 4000×g and the supernatant was pipetted into fresh 1.5 ml reaction tubes. Phytase activity was determined from each of these supernatants.

The supernatant was diluted 1:80 with a sodium acetate buffer (250 mM HAc-NaAc buffer, pH 4.5 containing 0.01% Tween20). Of this, 800 μl of phytate solution (7.5 mmol/1) was added to 400 μl. After incubation for 30 min at 37° C., the reaction was stopped and mixed thoroughly. This was followed by incubation at room temperature (1 min) and then centrifugation of the reaction mixture at 11000×g.

The enzyme activity was measured in triplicates in 96-well plates. The absorbance were measured at 415 nm in a plate reader (FLUOstar Omega, BMG Labtech).

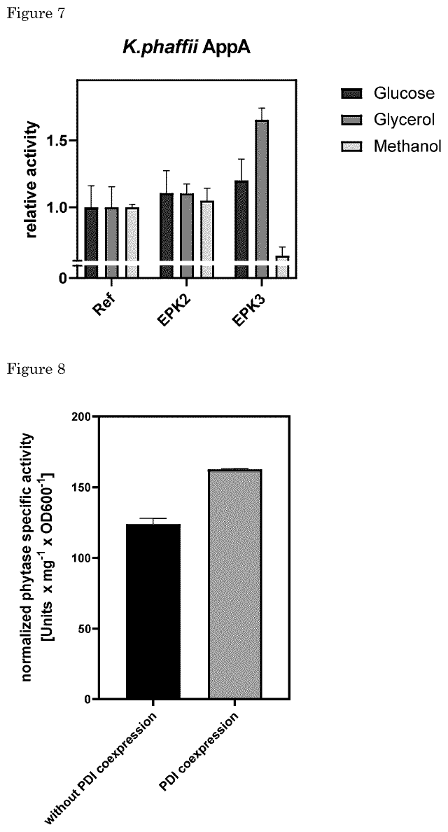

The measured activity was normalized to OD600 and the reference promoter GAP1 was set to 1 (FIG. 7).

In further experiments, the carbon source was exchanged to provide glycerol or methanol instead of glucose.

For other applications, it is not necessary that the promoters are very strong. Some of the promoters showed weaker fluorescence intensities compared to the reference promoter GAP1. These promoters are interesting for metabolic engineering, as being able to regulate multiple genes individually.

However, these individually designed promoters can also regulate the expression of auxiliary enzymes, such as PDI (B3VSN1). It is known that the enzyme PDI can increase the correct folding rate of recombinantly expressed proteins, especially in the presence of additional disulfide bridges. To increase the correct folding rate of the desired protein such as AppA, there are other auxiliary enzymes like ERV2 (C4R490), EUG1 (P32474), ERO1 (A0A1B2J869) and MPDI (C4QVB2).

Thus, co-expression with the PDI enzyme increases the secretion of appA phytase and apparently provides a lower misfolding rate (FIG. 8).

Furthermore, there is the possibility to express auxiliary enzymes that allow K. phaffii to utilize other carbon sources. For example, the invertase SUC2 (P00724) is an enzyme with which sucrose would be available as a carbon sources.

SEQUENCES

SEQ ID NO: 1 > EPK1
TGTGTTGGTGCTGGTGACTCATGGAATATGGGCGCGTGTCTTCCTGACCAAATGGTTTAGATGG
AGCGTAGAAAAGTTCGAAGACCTGCAAAACGTGCCTAACTGTCGCTGGATAGTGATGGAAAAGG
ACGAGACGACGCAGCGTTACGTCCTTCGCACGCATCTGAGCACGTGGTCAGAGCTTGAACAGAC
AAAAAGAGAGGAAGAAGTCAAAAGAGATGCCGGGAGAGAGTTTACATTCAACAGTACAGTGCCT
CTCACAGACGACGAGGTAAGACAGGTAGCCGACGCGGAGGCGCGAGCGAAAAACGAGCAGGTGC
AAAAATCGCTAAAAATAAGCGATTCCGTCAGACTCACTGCATCTGGGTCAGAGGGAGGCTTTAG
GACAGGAGCCATCTGTACAGAGGCTACGGAGTGTGGTGGCGGGTTCATGGGTGGCTCAAGCGGT
CTAAAAGCAAAGGTGCGCGGCCGTACGTTATTGTTTGTGTGGGTACGCGATAAATAAAAAATCA
GACAATCGGCTATGGGGGTGACATAAGCGATGAGCAAACTCTATAGCCTCGGCAAACCTGGGCG
TGCACGGATGTCGTCGGAGTGCAATTTTCCAGCGGACCAAAGTTCACCAGGAAAAAAATGACCC
AATATGGGCGGTGCCCAATGATCACACCAACAATTGGTCCACCCCTCCCCAATCTCTAATATTC
ACAATTCACCTCACTATAAATACCCCTGTCCTGCTCCCAAATTCTTTTTTCCTTCTTCCATCAG
CTACTAGCTTTTATCTTATTTACTTTACGAAA
SEQ ID NO: 2 > EPK2
TATTTTCACAATTGCACCCCAGCCAGACCGATAGCCGGCCGCAATCCGCCACCCACAACCGTCT
ACCTCCCACAGAACCCCGTCACTTCCACCCTTTTCCACCAGATCATATGTCCCAACTTGCCAAA
TTAAAACCGTGCGAATTTTCAAAATAAACTTTGGCAAAGAGGCTGCAAAGGAGGGGCTGGTGAG
GGCGTCTGGAAGTCGACCAGACACCGGGTTGGCGGCGTATTTGTGTCCCAAAAAACAGCCCCAA
TTGCCCCAATTGACCCCAAATTGACCCAGTAGCGGGCCCAACCCCGGCGAGAGCCCCCTTCACC
CCACATATCAAACCTCCCCCGGTTCCCACACTTGCCGTTAAGGGCGTAGGGTACTGCAGTCTGG
AATCTACGCTTGTTCAGACTTTGTACTAGTTTCCTAAAACATGCAATCGGCTGCCCCGCAACGG
GAAAAAGAATGACTTTGGCACTCTTCACCAGAGTGGGGTGTCCCGCTCGTGTGTGCAAATAGGC
TCCCACTGGTCACCCCGGATTTTGCAGAAAAACAGCAAGTTCCGGGGTGTCTCACTGGTGTCCG
CCAATAAGAGGAGCCTAACGTTCATGATCAAAATTTAACTGTTCTAACCCCTACTTGACAGCAA
TATATAAACAGAAGGAAGCTGCCCTGTCTTAAACCTTTTTTTTATCATCATTATTAGCTTACTT
TCATAATTGCGACTGGTTCCAATTGACAAGCTTTTGATTTTAACGACTTTTAACGACAACTTGA
GAAGATCAAAAAACAACTAATTATTCGAAACG
SEQ ID NO: 3 > EPK3
CGAAATATACCACATTGCCAGTTTATACAGATGGTTAAGGGTGAAAATCAACGTTACACCTTGA
CGACCCCATTATTACGATGGCGTGAAGGAGATGAAGACCGGGTAGAAGAAATAAGAAAAGCGGT
ACAGTTTAGGTCCGGAGATCTAGGGAAGGAGGCCTTAGCTTATATTGTAGCTGCTGAGAGAGAG
GCAGCTGCTGGAAGATCTGAAGGCCCTATCACGTATGATGATGGTGATGACCATTAGAGAACGC
CCAGAGATTGATAGCCAGTTCTTGGACAACCGAGTCTCTCGGAAAACAGCTTCTGGATATCTTC
CGCTGGCGGCGCAACGACGAATAATAGTCCCTGGAGGTGACGGAATATATATGTGTGGAGGGTA
AATCTGACAGGGTGTAGCAAAGGTAATATTTTCCTAAAACATGCAATCGGCTGCCCCGCAACGG
GAAAAAGAATGACTTTGGCACTCTTCACCAGAGTGGGGTGTCCCGCTCGTGTGTGCAAATAGGC
TCTGCTGGAGAGCTTCTTCTACGGCCCCCTTGCAGCAATGCTCTTCCCAGCATTACGTTGCGGG
TAAAACGGAGGTCGTGTACCCGACCTAGCAGCCCAGGGATGGAAAAGTCCCGGCCGTCGCTGGC
TATATAAGCGGGCGGACGCATGTCATGAGATTATTGGAAACCACCAGAATCGAATATAAAACGC
GAACACCTTTCCCAATTTTGGTTTCTCCTGACCCAAAGACTTTAAATTTAATTTATTTGTCCCT
ATTTCAATCAATTGAACAACTATCAAAACACA
SEQ ID NO: 4 > EPK4
CGAAATATACCACATTGCCAGTTTATACAGATGGTTAAGGGTGAAAATCAACGTTACACCTTGA
CGACCCCATTATTACGATGGCGTGAAGGAGATGAAGACCGGGTAGAAGAAATAAGAAAAGCGGT
ACAGTTTAGGTCCGGAGATCTAGGGAAGGAGGCCTTAGCTTATATTGTAGCTGCTGAGAGAGAG
GCAGCTGCTGGAAGATCTGAAGGCCCTATCACGTATGATGATGGTGATGACCATTAGAGAACGC
CCAGAGATTGATAGCCAGTTCTTGGACAACCGAGTCTCTCGGAAAACAGCTTCTGGATATCTTC
CGCTGGCGGCGCAACGACGAATAATAGTCCCTGGAGGTGACGGAATATATATGTGTGGAGGGTA
AATCTGACAGGGTGTAGCAAAGGTAATATTTTCCTAAAACATGCAATCGGCTGCCCCGCAACGG
GAAAAAGAATGACTTTGGCACTCTTCACCAGAGTGGGGTGTCCCGCTCGTGTGTGCAAATAGGC
TCCCACTGGTCACCCCGGATTTTGCAGAAAAACAGCAAGTTCCGGGGTGTCTCACTGGTGTCCG
CCAATAAGAGGAGCCGGCAGGCACGGAGTCTACATCAACTGTTCTAACCCCTACTTGACAGCAA
TATATAAACAGAAGGAAGCTGCCCTGTCTTAAACCTTTTTTTTATCATCATTATTAGCTTATTC
ACAATTCACCTCACTATAAATACCCCTGTCCTGCTCCCAAATTCTTTTTTCCTTCTTCCATCAG
CTACTAGCTTTTATCTTATTTACTTTACGAAA
SEQ ID NO: 5 > EPK5
CGAAATATACCACATTGCCAGTTTATACAGATGGTTAAGGGTGAAAATCAACGTTACACCTTGA
CGACCCCATTATTACGATGGCGTGAAGGAGATGAAGACCGGGTAGAAGAAATAAGAAAAGCGGT
ACAGTTTAGGTCCGGAGATCTAGGGAAGGAGGCCTTAGCTTATATTGTAGCTGCTGAGAGAGAG
GCAGCTGCTGGAAGATCTGAAGGCCCTATCACGTATGATGATGGTGATGACCATTAGAGAACGC
CCAGAGATTGATAGCCAGTTCTTGGACAACCGAGTCTCTCGGAAAACAGCTTCTGGATATCTTC
CGCTGGCGGCGCAACGACGAATAATAGTCCCTGGAGGTGACGGAATATATATGTGTGGAGGGTA
AATCTGACAGGGTGTAGCAAAGGTAATATTTTCCTAAAACATGCAATCGGCTGCCCCGCAACGG
GAAAAAGAATGACTTTGGCACTCTTCACCAGAGTGGGGTGTCCCGCTCGTGTGTGCAAATAGGC
TCCCACTGGTCACCCCGGATTTTGCAGAAAAACAGCAAGTTCCGGGGTGTCTCACTGGTGTCCG
CCAATAAGAGGAGCCGGCAGGCACGGAGTCTACATCAACTGTTCTAACCCCTACTTGACAGCAA
TATATAAACAGAAGGAAGCTGCCCTGTCTTAAACCTTTTTTTTATCATCATTATTATAAAAGGC
GAACACCTTTCCCAATTTTGGTTTCTCCTGACCCAAAGACTTTAAATTTAATTTATTTGTCCCT
ATTTCAATCAATTGAACAACTATCAAAACACA
SEQ ID NO: 6 > EPK6
GTTCCGGCTCCGATTTCTCGGCCAAGTGCCGACTCCACAAGACCGAGGAAGGTAAACTCTCTGT
TCACGTGGGGCTGAAGAAAGGCCAGCTCACGGTGGTGGAGTTGCAATGGAGGCGGCAGCTGAAT
AGACCTTGCAAGTAAGGGGTACCTGACGGGAATGTGGGAATATGGGACACAAATTGGGGGGCAA
ACTTGCAACATGCGTCCAGTGTACCCCGGATAACACCATTAGTGTGGCTAATAGCGAGGATGGG
GACTTGGAGGGATGGGTTCGCAGGGATGGACTCGGAGGGATGGACGCGAATGGCGTGGAGGGCT
CGGATGGCGCGGAGGGTTCGGAGATGGGTCCAGGGCCAAAAATCCGGTTTAAAAAGGTGGATAT
GGTCGATTGTAGTGGAAGCTCGTGATCCTACATGCTCACGAAACCCATCATCATGCAATCCACA
TTAAAGGAAGGGAAAAGGATATTGAACTTTTGACTATTTAGTATAAATGAAAACTACTTTGTAA
GCTTGGACAGAGGAATAATTTCTGATTCGTGCTTCTGCTTCTACTGACTTGCAAACCAGTAAAC
AAGTTACTGCATTAATCAGTAATAATCTGCGTATAGCGAAACTTGTAACCCTACTTGACAGCAA
TATATAAACAGAAGGAAGCTGCCCTGTCTTAAACCTTTTTTTTATCATCATTATTAGCTTACTT
TCATAATTGCGACTGGTTCCAATTGACAAGCTTTTGATTTTAACGACTTTTAACGACAACTTGA
GAAGATCAAAAAACAACTAATTATTCGAAACG
SEQ ID NO: 7 > EPK7
AGGTCTTTAGTCAGAGGCAGGAACAGCCGTCAAGGGGGCATAAGACTACGGTCATCCCCATCTG
CCTCTTCGTCCAGCCTTGCCAACAGGGAGTTCTTCAGAGACATGGAGGCTCAAAACGAAATTAT
TGACAGCCTAGACATCAATAGTCATACAACAGAAAGCGACCACCCAACTTTGGCTGGGGGTAGC
GTATAAACAATGCATACTTTGTACGTTCAAAATACAATGCAGTAGATATATTTATGCATATTAC
ATATAATACATATCACATAGGAATGGACAGACAACTATACCAGCATGGATCTCTTGTATCGGTT
CTTTTCTCCCGCTCTCTCGCAATAACAATGAACACTGGGTCAATCATAGCCTACACAGGTGAAC
AGAGTAGCGTTTATACAGGGTTTATACGGTGATTCCTACGGCAAAAATTTTTCATTTCTAAAAA
GAAAAAGAAAAATTTTTCTTTCCAACGCTAGAAGGAAAAGAAAAATCTAATTAAATTGATTTGG
TGATTTTCTGAGAGTTCCCACACTTGCCGTTAAGGGCGTAGGGTACTGCAGTCTGGAATCTACG
CTTGTTCAGACTTTGTACTAGTTTCTTTGTCTGGCCATCCGGGTAACCCATGCCGGACGCTATA
TAAGCTACTGAAAATTTTTTTGCTTTGTGGTTGGGACTTTAGCCAAGGGTATAAAAGACCACCG
TCCCCGAATTACCTTTCCTCTTCTTTTCTCTCTCTCCTTGTCAACTCACACCCGAAATCGTTAA
GCATTTCCTTCTGAGTATAAGAATCATTCAAA
SEQ ID NO: 8 > EPK8
TTCGCTCCCACACTACACCGTAATACCACGTCACTCTCATTGCAGGTTACCCTGCCCGTAGTCG
CTCGATCCACCTCCTCCTTCTCTCGTGTGTGCAGCAAAGAGGCAGAGATGGAGCCCGTATGGTG
AATTAAAACCGTGCGAATTTTCAAAATAAACTTTGGCAAAGAGGCTGCAAAGGAGGGGGGGGTG
AGGGCGTCTGGAAGTCGACCAGAGACCGGGTTGGCGGCGTATTTGTGTCCCAAAAAACAGCCCC
AATTGCCCCAAGTCCACCACCACTGGACACTTGATTTACAAGTGCGGTGGTATGGATAAGCGAA
CCATTGAAAAGTTTGAGAAGGAAGCCGATGAGCTTGGAAAGGGTTCTTTCAAGTACGCTTGGGT
TCTTGACAAGTTGAAGGCTGAGCGAGAGCGAGGTATCACCATTGATATTGCTCTCTGGAAGTTC
GAGACCCCCAAGTACTACGTTACCATTATTGATGCTCCCGGTCACCGAGATTTCATCAAGAATA
TGATTACCGGTACTTCCCCACACTTGCCGTTAAGGGCGTAGGGTACTGCAGTCTGGAATCTACG
CTTGTTCAGACTTTGTACTAGTTTCTTTGTCTGGCCATCCGGGTAACCCATGCCGGACGCTATA
TAAGCTACTGAAAATTTTTTTGCTTTGTGGTTGGGACTTTAGCCAAGGGTATAAAAGACCACCG
TCCCCGAATTACCTTTCCTCTTCTTTTCTCTCTCTCCTTGTCAACTCACACCCGAAATCGTTAA
GCATTTCCTTCTGAGTATAAGAATCATTCAAA
SEQ ID NO: 9 > EPK9
AGGTCTTTAGTCAGAGGCAGGAACAGCCGTCAAGGGGGCATAAGACTACGGTCATCCCCATCTG
CCTCTTCGTCCAGCCTTGCCAACAGGGAGTTCTTCAGAGACATGGAGGCTCAAAACGAAATTAT
TGACAGCCTAGACATCAATAGTCATACAACAGAAAGCGACCACCCAACTTTGGCTGGGGGTAGC
GTATAAACAATGCATACTTTGTACGTTCAAAATACAATGCAGTAGATATATTTATGCATATTAC
ATATAATACATATCACATAGGAAGCAACACTTGATTTACAAGTGCGGTGGTATGGATAAGCGAA
CCATTGAAAAGTTTGAGAAGGAAGCCGATGAGCTTGGAAAGGGTTCTTTCAAGTACGCTTGGGT
TCTTGACAAGTTGAAGGCTGAGCGAGAGCGAGGTATCACCATTGATATTGCTCTCTGGAAGTTC
GAGACCCCCAAGTACTACGTTACCATTATTGATGCTCCCGGTCACCGAGATTTCATCAAGAATA
TGATTACCGGTACTTCCCCACACTTGCCGTTAAGGGCGTAGGGTACTGCAGTCTGGAATCTACG
CTTGTTCAGACTTTGTACTAGTTTCTTTGTCTGGCCATCCGGGTAACCCATGCCGGACGCTATA
TAAGCTACTGAAAATTTTTTTGCTTTGTGGTTGGGACTTTAGCCAAGGGTATAAAAGACCACCG
TCCCCGAATTACCTTTCCTCTTCTTTTCTCTCTCTCCTTGTCAACTCACACCCGAAATCGTTAA
GCATTTCCTTCTGAGTATAAGAATCATTCAAA
SEQ ID NO: 10 > EPK10
GGTATTTTCACAATTGCACCCCAGCCAGACCGATAGCCGGCCGCAATCCGCCACCCACAACCGT
CTACCTCCCACAGAACCCCGTCACTTCCACCCTTTTCCACCAGATCATATGTCCCAACTTGCCA
AATTAAAACCGTGCGAATTTTCAAAATAAACTTTGGCAAAGAGGCTGCAAAGGAGGGGGGGGTG
AGGGCGTCTGGAAGTCGACCAGAGACCGGGTTGGCGGCGTATTTGTGTCCCAAAAAACAGCCCC
AATTGCCCCAATTGACCCCAAATTGACCCAGTAGCGGGCCAGCGTATCCCAGCCTAGTGTATCC
CAGCCTAGCCTAGCCTAGGCCAAACCTAGCCCTCTCTAGCCTAGCGCCCAGCAGAAACACCGAT
GAAGCAAAGAAGTAACAGCAGGAAAGAAAAACAAACACAACAAAAAAAAACAAGCAGCATAGCA
TCAACAGAAATTTCTAAAGAGAACCAAATTCACCCCAGAAACAACCGCACAAATACGACATCCA
TCCACCTTTCTTTTATCCCACACTTGCCGTTAAGGGCGTAGGGTACTGCAGTCTGGAATCTACG
CTTGTTCAGACTTTGTACTAGTTTCTTTGTCTGGCCATCCGGGTAACCCATGCCGGACGCTATA
TAAGCTACTGAAAATTTTTTTGCTTTGTGGTTGGGACTTTAGCCAAGGGTATAAAAGACCACCG
TCCCCGAATTACCTTTCCTCTTCTTTTCTCTCTCTCCTTGTCAACTCACACCCGAAATCGTTAA
GCATTTCCTTCTGAGTATAAGAATCATTCAAA
SEQ ID NO: 11 > EPK11
AGGTCTTTAGTCAGAGGCAGGAACAGCCGTCAAGGGGGCATAAGACTACGGTCATCCCCATCTG
CCTCTTCGTCCAGCCTTGCCAACAGGGAGTTCTTCAGAGACATGGAGGCTCAAAACGAAATTAT
TGACAGCCTAGACATCAATAGTCATACAACAGAAAGCGACCACCCAACTTTGGCTGGGGGTAGC
GTATAAACAATGCATACTTTGTACGTTCAAAATACAATGCAGTAGATATATTTATGCATATTAC
ATATAATACATATCACATAGGAAGCAACAGGCGCCATCCCAGCGTATCCCAGCCTAGTGTATCC
CAGCCTAGCCTAGCCTAGGCCAAACCTAGCCCTCTCTAGCCTAGCGCCCAGCAGAAACACCGAT
GAAGCAAAGAAGTAACAGCAGGAAAGAAAAACAAACACAACAAAAAAAAACAAGCAGCATAGCA
TCAACAGAAATTTCTAAAGAGAACCAAATTCACCCCAGAAACAACCGCACAAATACGACATCCA
TCCACCTTTCTTTTATCCCACACTTGCCGTTAAGGGCGTAGGGTACTGCAGTCTGGAATCTACG
CTTGTTCAGACTTTGTACTAGTTTCTTTGTCTGGCCATCCGGGTAACCCATGCCGGACGCTATA
TAAGCTACTGAAAATTTTTTTGCTTTGTGGTTGGGACTTTAGCCAAGGGTATAAAAGACCACCG
TCCCCGAATTACCTTTCCTCTTCTTTTCTCTCTCTCCTTGTCAACTCACACCCGAAATCGTTAA
GCATTTCCTTCTGAGTATAAGAATCATTCAAA
SEQ ID NO: 12 > EPK12
AGGAAACCTCGATGATTCTCCCGTTCTTCCATGGGCGGGTATCGCAAAATGAGGAATTTTTCAA
ATTTCTCTATTGTCAAGACTGTTTATTATCTAAGAAATAGCCCAATCCGAAGCTCAGTTTTGAA
AAAATCACTTCCGCGTTTCTTTTTTACAGCCCGATGAATATCCAAATTTGGAATATGGGGTACT
CTATCGGGACTGCAGATAATATGACAACAACGCAGATTACATTTTAGGTAAGGCATAAATTCTA
CAGGCACCTGCGAGGCAAGCAATCTACTAATGTTTATTTTTCGTCCAACCTAATTGTGGTTTCA
AAGCGCTATCAGGTGGGGGGTAAGAGGAATGTGAGTGGAAAGCGAAAATAACTGGCAGCTGGGG
TCAGATCCCGTGATGCCACCTCTTGTGGTATTTTGAAACGCGTGTTGCGATTGGCCGCGAGAAC
GGAAAGGAATATATTTACTGCCGATCGCATTTTGGCCTCAAATAAATCTTGAGCTTTTGGACAT
AGGTCTGTGGACACATGTCATGTTAGTGTACTTCAATCGCCCCCTGGATATAGCCCCGACAATA
GGCCGTGGCCTCATTTTTTTGCCTTCCGCACATTTCCATTGCTCGGTACCCACACCTTGCTTCT
CCTGCACTTGCCAACCTTAATACTGGTTTACATTGACCAACATCTTACAAGCGGGGGGCTTGTC
TAGGGTATATATAAACAGTGGCTCTCCCAATCGGTTGCCAGTCTCTTTTTTCCTTTCTTTCCCC
ACAGATTCGAAATCTAAACTACACATCACACA
SEQ ID NO: 13 > EPK13
CTTTCATAGAATATACTATGATCCGTTAGCAATTTTTGTCGTCACCTTGTTCCAATTCCATTGC
CATCTGTTTAGCTCCAGTTTTTCAGTTCACTCACTCATGATGGAAGTACAGGAACGAAAAGAAA
AATGTTTCTGAAAAAAAAAAGATGTTGGGGGGACAATTTGTGGAATCCGCCTGGATAAGCTCGA
GGGAAAACTGGAAGCTAGGAAGTTTGTGCACAAAGAAAGAGTGAATACAAGAAGCCAATAGCCC
GGCGTCCTAAATTGTACATTTGTGTCACATTATGAATTACAGGAAGTCAGAAAACAGGCAGCAC
ATGTCTCGCACATGCATGTCCATCAGACGAGACATTATGAGACATGCACGCGTGTGAGAGACAT
AGCAAAAGTCTCTCCAGTACACACAGAAAGACACGTTCACAATCCAGGCACCCCACAGAGAAAA
AAAAAAGAAGAAGCCCGGAAGCTGGCACGCCATCATCAACCACCGCTCGGTTTACACGCATCCC
AACTGTCTTTTTTTCTTTTCCTCTCGTAGGGTTGGGAATCCAGTATTGTGGGCTAATGAACTGA
GTCACATAATGTGGTTATGTTCCAATATAGGTACCACCTTTGTTCAAGATTTAGTTTTCTAATT
GAATATAAATACAGAGGTTATTTCAACCTAATTGAGATTAAGGAGAGACTTATTTTACTATAGT
ATATATTTATTTATAATTACTTATTGTTACTCCAATCCCCAAGTAGATTAGATTTAATCAATCA
CACACAAACATCAAAACAACAAATTAACAAAA
SEQ ID NO: 14 > EPK14
TTTTTTTATTTTTTGTCCCTCCGGGATGGCAAGAGGGACAAAGAAGAATCTTCGTTCTTCTTTC
TTGTTCTCAACTTCCCAGCTTCCGTGTGATTACCCTCCGGGACAACAGAAAAACTGGCATTCGG
TATCCAGGGAATCTGCTGAGAAGGAAAGAAAACGAAAAAAAAATTGTACATTTGTGTCACATTA
TGAATTACAGGAAGTCAGAAAACAGGCAGCACATGTCTCGCACATGCATGTCCATCAGACGAGA
CATTATGAGACATGTCACATTTTTTATTTTTTAATTTTTCATTACGCAGCAAACATGCAGCATC
CACTAACTTCAGAGATTCCTGTAAGATAAGTGGTTCGTTATTTTCCGGATTCCAATTTTGGTGG
TGCTCCGAAAAGTGGAAGCTCGTGATCCTACATGCTCACGAAACCCATCATCATGCAATCCACA
TTAAAGGAAGGGAAAAGGATATCTGCTGGATAATTTTCAGAGGCAACAAGGAAAAATTAGATGG
CAAAAAGTCGTCTTTCAAGGAAAAATCCCCACCATCTTTCGAGATCCCCTGTAACTTATTGGCA
ACTGAAAGAATGAAAAGGAGGAAAATACAAAATATACTAGAACTGAAAAAATAAAAGTATAAAT
AGAGAAGATATATGCCAATACTTCACAATGTTCGAATCTATTCTTCATTTGCAGCTATTGTAAA
ATAATAAAACATCAAGAACAAACAAGCTCAACTTGTCTTTTCTAAGAACAAAGAATAAACACAA
AAACAAAAAGTTTTTTTAATTTTAATCAAAAA
SEQ ID NO: 15 > EPK15
AAAACGAAGATTAAGATAAAGTTGGGTAAAATCCGGGGTAAGAGGCAAGGGGGTAGAGAAAAAA
AAACCGGAGTCATTATATACGATACCGTCCAGGGTAAGACAGTGATTTCTAGCTTCCACTTTTT
TCAATTTCTTTTTTTCGTTCCAAATGGCGTCCACCCGTACATCCGGAATCTGACGGCACAAGAG
CCGATTAGTGGAAGCCACGGTTACGTGATTGCGGTTTTTTTTTCCTACGTATAACGCTATGACG
GTAGTTGAAGTTCATCAAAGTGTTGGACAGACAACTATACCAGCATGGATCTCTTGTATCGGTT
CTTTTCTCCCGCTCTCTCGCAATAACAATGAACACTGGGTCAATCATAGCCTACACAGGTGAAC
AGAGTAGCGTTTATACAGGGTTTATACGGTGATTCCTACGGCAAAAATTTTTCATTTCTAAAAA
AAAAAAGAAAAATTTTTTGCTTCTGCTGGATAATTTTCAGAGGCAACAAGGAAAAATTAGATGG
CAAAAAGTCGTCTTTCAAGGAAAAATCCCCACCATCTTTCGAGATCCCCTGTAACTTATTGGCA
ACTGAAAGAATGAAAAGGAGGAAAATACAAAATATACTAGAACTGAAAAAAAAAAAGTATAAAT
AGAGAAGATATATGCCAATACTTCACAATGTTCGAATCTATTCTTCATTTGCAGCTATTGTAAA
ATAATAAAACATCAAGAACAAACAAGCTCAACTTGTCTTTTCTAAGAACAAAGAATAAACACAA
AAACAAAAAGTTTTTTTAATTTTAATCAAAAA
SEQ ID NO: 16 > EPK16
TTGCTGGGATCACCCATACATCACTCTGTTTTGCCTGACCTTTTCCGGTAATTTGAAAACAAAC
CCGGTCACGAAGCGGAGATCCGGCGATAATTACCGCAGAAATAAACCCATACACGAGAAGTAGA
ACCAGCCGCACATGGCCGGAGAAACTCCTGCGAGAATTTCGTAAACTCGCGCGCATTGCATCTG
TATTTCCTAATGCGGCACTTCCAGGCCTCGAGAACTCTGACATGCTTTTGACAGGAATAGACAT
TTTCAGAATGTTATCCATATGCCTTTCGGGTTTTTTTCCTTCCTTTTCCATCATGAAAAATCTC
TCGAGAACGTTTATCCATTGCTTTTTTGTTGTCTTTTTCCCTCGTTCACAGAAAGTCTGAAGAA
GCTATAGTAGAACTATGAGCTTTTTTTGTTTCTGTTTTCCTTTTTTTTCTCGTAGGAACAATTT
CGGGCCCCTGCGTGTTCTTCTGAGGTTCATCTTTTACATTTGCTTCTGCTGGATAATTTTCAGA
GGCAACAAGGAAAAATTAGATGGCAAAAAGTCGTCTTTCAAGGAAAAATCCCCACCATCTTTCG
AGATCCCCTGTAACTTATTGGCAACTGAAAGAATGAAAAGGAGGAAAATACAATAATGTAAATT
TTTTTTGAATATAAAAGGAGATTGAAAAATTTTTTCTAGCAGAAATGTTTTCAAGTTTTAATTG
CAAGTTTCGTTTGAGTATTCAGTTGTATTTTAGTTGATTTGTAGTTTATTTACTAGTATTCTCA
TAGTTCTAACTCCAAGAGAAGTAACATTAAAG
SEQ ID NO: 17 > EPK17
AAAACGAAGATTAAGATAAAGTTGGGTAAAATCCGGGGTAAGAGGCAAGGGGGTAGAGAAAAAA
AAACCGGAGTCATTATATACGATACCGTCCAGGGTAAGACAGTGATTTCTAGCTTCCACTTTTT
TCAATTTCTTTTTTTCGTTCCAAATGGCGTCCACCCGTACATCCGGAATCTGACGGCACAAGAG
CCGATTAGTGGAAGCCACGGTTACGTGATTGCGGTTTTTTTTTCCTACGTATAGCAACCTGTAT
TCGGTCATTGATGCATGCATGTGCCGTGAAGCGGGACAACCAGAAAAGTCGTCTATAAATGCCG
GCACGTGCGATCATCGTGGCGGGGTTTTAAGAGTGCATATCACAAATTGTCGCATTACCGCGGA
ACCGCCAGATATTCATTACTTGACGCAAAAGCGTTTGAAATAATGACGAAAAAGAAGGAAGAAA
AAAAAAGAAAAATACCGCTTCTAGGCGGGTTATCTACTGATCCGAGCTTCGCTGGCGGGAGAAG
GAAAAAAAAAATTTTTTTTTTCCTTCTGTTTAGTACTGGAACATTGAGAAGGCGTGTCAATTTT
GAATAATTAGAGTGGTCAAAAAAATTTTTTTTGCTTGGGATACCCTTTTTCGATAATGTAAATT
TTTTTTGAATATAAAAGGAGATTGAAAAATTTTTTCTAGCAGAAATGTTTTCAAGTTTTAATTG
CAAGTTTCGTTTGAGTATTCAGTTGTATTTTAGTTGATTTGTAGTTTATTTACTAGTATTCTCA
TAGTTCTAACTCCAAGAGAAGTAACATTAAAG
SEQ ID NO: 18 > EPK18
GGTCACAATGTTTAAAGCTCTGTCGGTAAATTGTGATTGATCCATGATTAGCAAAATTGCAGTT
GTAGGGAAAGCAATTTATACAGCGTACAGACATGAGACGCTTGTGATGGTCTGGTGATCTAGAG
CCGCACAGAAACTTCTAGCAATATCTGGTGTGTCTGGGTGGTTGCTGGTGTCTGGTCTGCATAA
CTTGGCCTGAGGATGCTTATGTAATATGGCCTTCCTGGTGCATGGAAGTGGCTGGTGGCAAAAA
AAAGTCTGTGAGCCTCCCAAGCAATCTACTAATGTTTATTTTTCGTCCAACCTAATTGTGGTTT
CAAAGCGCTATCAGGTGGGGGGTAAGAGGAATGTGAGTGGAAAGCGAAAATAACTGGCAGCTGG
GGTCAGATCCCGTGATGCCACCTCTTGTGGTATTTTGAAACGCGTGTTGCGATTGGCCGCGAGA
ACGGAAAGGAATATATTTACTGCCGATCGCATTTTGGCCTCAAATAAATCTTGAGCTTTTGGAC
ATAGATTATATGTTCTTTCTTGGAAGCTCTTTCAGCTAATAGTGAAGTGTTTCCTACTAAGGAT
CGCCTCCAAACGTTCCAACTACGGGCGGAGGTTGCAAAGAAAACGGGTCTCTCAGCGAATTGTT
CTCATCCATGAGTGAGTCCTCTCCGTCCTTTCCTCGCGCCTGCCTAATCCATCGTCCAACAGAG
AGGTCGCTCTCCTTATATATATAGTTGATCCCCCTTTTTTTCTACCCTTGCAATTTTTTTTTGG
GACCAAAGAAAAGAAACAAGACTGATACAAAA
SEQ ID NO: 19 > EPK19
GTTAGTAGCAACCTGAACTCGGTCATTGATGCATGCATGTGCCGTGAAGCGGGACAACCAGAAA
AGTCGTCTATAAATGCCGGCACGTGCGATCATCGTGGGGGGTTTTAAGAGTGCATATCACAAA
TTGTCGCATTACCGCGGAACCGCCAGATATTCATTACTTGACGCAAAAGCGTTTGAAATAATGA
CGAAAAAGAAGGAAGAAAAAAAAAGAAAAATACCGCTTCTAGGCGGGTTATCTACTGATCCGAG
CTTCCACTAGGATAGCACCCAAACACCTGCATATTTGGACGACCTTTACCAAATGAGAACCCTG
TATTTCCTTGGCCAGAGCCTGAGAAACTGAACCCTGATCCAGACATTATCAATACCTTGGGTTA
TTAGTAGTGTCCGTTATTTTTCTGTTTAGGTTACGATTTTGCCAGATTTTTTGGGAGGAGGGAA
ACAAAAGAACCAGTGCTACACGACCTTTAAGTGCCATCAGGCATCCTGTTTTCTCGACCTCATC
TCATCACATCCGTCAGTCTGAGCTTTCAGTTCTCAGTTTTCGATTGACTCTTGCCCTGCTGCGC
GCACACCATACCCTGGCTCCCTCTCATGCTTCTGGCGTTACCCCGGAATCGTACATCCATGCCG
CGAATCCCGGACAGGACTCAGACGGATTTCACTATTTGGGCGGGCTTGCTCCGTCTGTCCAAGG
CAACATTTATATAAGGGTCTGCATCGCCGGCTCAATTGAATCTTTTTTCTTCTTCTCTTCTCTA
TATTCATTCTTGAATTAAACACACATCAACA
SEQ ID NO: 20 > EPK20
AGGTCTTTAGTCAGAGGCAGGAACAGCCGTCAAGGGGGCATAAGACTACGGTCATCCCCATCTG
CCTCTTCGTCCAGCCTTGCCAACAGGGAGTTCTTCAGAGACATGGAGGCTCAAAACGAAATTAT
TGACAGCCTAGACATCAATAGTCATACAACAGAAAGCGACCACCCAACTTTGGCTGGGGGTAGC
GTATAAACAATGCATACTTTGTACGTTCAAAGGTTTGGAACAACACTAAACTACCTTGCGGTAC
TACCATTGACACTACACATCCTTAATTCCAATCCTGTCTGGCCTCCTTCACCTTTTAACCATCT
TGCCCATTCCAACTCGTGTCAGATTGCGTATCAAGTGAAAAAAAAAAAATTTTAAATCTTTAAC
CCAATCAGGTAATAACTGTCGCCTCTTTTATCTGCCGCACTGCATGAGGTGTCCCCTTAGTGGA
AAAGAATACTGAGCCAACCCTGGAGGACAGCAAGGGAAAAATACCTACAACTTGCTTCATAATG
GTCGTAAAACCCGGTTCCCACACTTGCCGTTAAGGGCGTAGGGTACTGCAGTCTGGAATCTACG
CTTGTTCAGACTTTGTACTAGTTTCTTTGTCTGGCCATCCGGGTAACCCATGCCGGACGCTATA
TAAGCTACTGAAAATTTTTTTGCTTTGTGGTTGGGACTTTAGCCAAGGGTATAAAAGACCACCG
TCCCCGAATTACCTTTCCTCTTCTTTTCTCTCTCTCCTTGTCAACTCACACCCGAAATCGTTAA
GCATTTCCTTCTGAGTATAAGAATCATTCAAA
SEQ ID NO: 21 > EPK21
CGAAATATACCACATTGCCAGTTTATACAGATGGTTAAGGGTGAAAATCAACGTTACACCTTGA
CGACCCCATTATTACGATGGCGTGAAGGAGATGAAGACCGGGTAGAAGAAATAAGAAAAGCGGT
ACAGTTTAGGTCCGGAGATCTAGGGAAGGAGGCCTTAGCTTATATTGTAGCTGCTGAGAGAGAG
GCAGCTGCTGGAAGATCTGAAGGCCCTATCACGTATGATGATGGTGATGACCATTAGAGAACGC
CCAGAGATTGATAGCCAGTTCTTGGACAACCGAGTCTCTCGGAAAACAGCTTCTGGATATCTTC
CGCTGGCGGCGCAACGACGAATAATAGTCCCTGGAGGTGACGGAATATATATGTGTGGAGGGTA
AATCTGACAGGGTGTAGCAAAGGTAATATTTTCCTAAAACATGCAATCGGCTGCCCCGCAACGG
GAAAAAGAATGACTTTGGCACTCTTCACCAGAGTGGGGTGTCCCGCTCGTGTGTGCAAATAGGC
TCCCACTGGTCACCCCGGATTTTGCAGAAAAACAGCAAGTTCCGGGGTGTCTCTGGAATCTACG
CTTGTTCAGACTTTGTACTAGTTTCTTTGTCTGGCCATCCGGGTAACCCATGCCGGACGCTATA
TAAGCTACTGAAAATTTTTTTGCTTTGTGGTTGGGACTTTAGCCAAGGGTATAAAACACCACCG
TCCCCGAATTACCTTTCCTCTTCTTTTCTCTCTCTCCTTGTCAACTCACACCCGAAATCGTTAA
GCATTTCCTTCTGAGTATAAGAATCATTCAAA
SEQ ID NO: 22 > EPK22
GGTGGCTCTTCTTGTTTTTCTTACTGAAAAGAGACTGGAACCGAGCTCTCGAAGAAGGCCGGTA
GGTGCGTTTTCGCGGCGACCGTCGCACTATGTCCCGCACGCGCCGCTTGGTAGACTGGCCAGCG
TCGTCCTGGCTGTGTGCATCGCTTTTGTTGAGCTCGTGTTGGAATTTCAGTATAAGCGATTCCG
TCAGACTCACTGCATCTGGGTCAGAGGGAGGCTTTAGGACAGGAGCCATCTGTACAGAGGCTAC
GGAGTGTGGTGGCGGGTTCATGGGTGGCTCAAGCGGTCTAAAAGCAAAGGTGCGCGGCCGTACG
TTATTGTTTGCACCCGTGTACATAAGCGTGAAATCACCACAAACTGTGTGTATCAAGTACATAG
TGACATTTAAATAATAGCAAGAACAACAATAATAGTAGCGCTACTGGAAGCACCACGTAATAGT
GGAAAAGAACTGGAAAAACCGCTATAAGATGCATACTCCGGCGGTCTTACGCGGAGATACAAGC
TCTGCTGGAGAGCTTCTTCTACGGCCCCCTTGCAGCAATGCTCTTCCCAGCATTACGTTGCGGG
TAAAACGGAGGTCGTGTACCCGACCTAGCAGCCCAGGGATGGAAAAGTCCCGGCCGTCGCTGGC
TATATAAGCGGGCGGACGCATGTCATGAGATTATTGGAAACCACCAGAATCGAATATAAAACGC
GAACACCTTTCCCAATTTTGGTTTCTCCTGACCCAAAGACTTTAAATTTAATTTATTTGTCCCT
ATTTCAATCAATTGAACAACTATCAAAACACA
SEQ ID NO: 23 > EPK23
CCACCGCAGACGCCCACCTCGTTAGCGTCCATTGCGATCCTCTCGGTACATTTGGTTACATTTT
GCGACAGGTTGAAATGAATCGGCCGACGCTCGGTAGTCGGAAAGAGCCGGGACCGGCCGGCGAG
CATAAACCGGACGCAGTAGGATGTCCTGCACGGGTCTTTTTGTGGGGTGTGGAGAAAGGGGTGC
TTGGAGATGGAAGCCGGTAGAACCGGGCTGCTTGGGGGGATTTGGGGCCGCTGGGCTCCAAAGA
GGGGTAGGCATTTCGTTGGGGTTACGTAATTGCGGCATTTGGGTCCTGCGCGCATGTCCCATTG
GTCAGAATTGCACCCGTGTACATAAGCGTGAAATCACCACAAACTGTGTGTATCAAGTACATAG
TGACATTTAAATAATAGCAAGAACAACAATAATAGTAGCGCTACTGGAAGCACCACGTAATAGT
GGAAAAGAACTGGAAAAACCGCTATAAGATGCATACTCCGGCGGTCTTACGCGGAGATACAAGC
TCTGCTGGAGAGCTTCTTCTACGGCCCCCTTGCAGCAATGCTCTTCCCAGCATTACGTTGCGGG
TAAAACGGAGGTCGTGTACCCGACCTAGCAGCCCAGGGATGGAAAAGTCCCGGCCGTCGCTGGC
TATATAAGCGGGCGGACGCATGTCATGAGATTATTGGAAACCACCAGAATCGAATATAAAACGC
GAACACCTTTCCCAATTTTGGTTTCTCCTGACCCAAAGACTTTAAATTTAATTTATTTGTCCCT
ATTTCAATCAATTGAACAACTATCAAAACACA
SEQ ID NO: 24 > EPK24
TTTTTTTATTTTTTGTCCCTCCGGGATGGCAAGAGGGACAAAGAAGAATCTTCGTTCTTCTTTC
TTGTTCTCAACTTCCCAGCTTCCGTGTGATTACCCTCCGGGACAACAGAAAAACTGGCATTCGG
TATCCCAGGAATCTGCTGAGAAGGAAAGAAAACGAAAAAAAAATTGTACATTTGTGTCACATTA
TGAATTACAGGAAGTCAGAAAACAGGCAGCACATGTCTCGCACATGCATGTCCATCAGACGAGA
CATTATGAGACATGCACGCGTGTGAGAGACATAGCAAAAGTCTCTCCAGTACACACAGAAAGAC
ACGTTCACAATCCAGGCACCCCACAGAGAAAAAATAAAGAAGAAGCCCGGAAGCTGGCACGCCA
TCATCAACCACCGCTCGGTTTACACGCATCCCAACTGTCTTTTTTTTCTGGAATCCTATAATTT
CGGGCCCCTGCGTGTTCTTCTGAGGTTCATCTTTTACATTTGCTTCTGCTGGATAATTTTCAGA
GGCAACAAGGAAAAATTAGATGGCAAAAAGTCGTCTTTCAAGGAAAAATCCCCACCATCTTTCG
AGATCCCCTGTAACTTATTGGCAACTGAAAGAATGAAAAGGAGGAAACCCCTACTTGACAGCAA
TATATAAACAGAAGGAAGCTGCCCTGTCTTAAACCTTTTTTTTATCATCATTATTAGCTTACTT
TCATAATTGCGACTGGTTCCAATTGACAAGCTTTTGATTTTAACGACTTTTAACGACAACTTGA
GAAGATCAAAAAACAACTAATTATTCGAAACG
SEQ ID NO: 25 > EPK25
CACATGTATAGTACGTTGCACATAGTCTACAATATTCAGCATTCAGCATTCAGTATACAGCATA
TGGCTAAATGATCACAAATGTGATTGATGATTTGACACGACTAGAAAAGAGAACGAAAAAGGGA
AATTCCATGTCACGTGCGTTGGCACGTGACATGGAATATCGAAGAAAGAAAAAAAAAACGATCT
CGTCCTAGTGGAAGCCCAGAGTCTGGTCCCCCCGGAGTCTTCCCAAAACAAGAAGCTGACACAT
GTTGACACAGTTCATCAAAGTGTTGGACAGACAACTATACCAGCATGGATCTCTTGTATCGGTT
CTTTTCTCCCGCTCTCTCGCAATAACAATGAACACTGGGTCAATCATAGCCTACACAGGTGAAC
AGAGTAGCGTTTATACAGGGTTTATACGGTGATTCCTACGGCAAAAATTTTTCATTTCTAAAAA
AAAAAAGAAAAATTTTTTGCTTCTGCTGGATAATTTTCAGAGGCAACAAGGAAAAATTAGATGG
CAAAAAGTCGTCTTTCAAGGAAAAATCCCCACCATCTTTCGAGATCCCCTGTAACTTATTGGCA
ACTGAAAGAATGAAAAGGAGGAAAATACAAAATATACTAGAACTGAAAAAAAAAAAGTATAAAT
AGAGAAGATATATGCCAATACTTCACAATGTTCGAATCTATTCTTCATTTGCAGCTATTGTAAA
ATAATAAAACATCAAGAACAAACAAGCTCAACTTGTCTTTTCTAAGAACAAAGAATAAACACAA
AAACAAAAAGTTTTTTTAATTTTAATCAAAAA
SEQ ID NO: 26 > EPK26
AATGAGGTCTTTAGTCAGAGGCAGGAACAGCCGTCAAGGGGGCATAAGACTACGGTCATCCCCA
TCTGCCTCTTCGTCCAGCCTTGCCAACAGGGAGTTCTTCAGAGACATGGAGGCTCAAAACGAAA
TTATTGACAGCCTAGACATCAATAGTCATACAACAGAAAGCGACCACCCAACTTTGGCTGATAA
TAGCGTATAAACAATGCATACTTTGTACGTTCAAAATACAATGCAGTAGATATATTTATGCATA
TTACATATAATACATCAAAGTGTTGGACAGACAACTATACCAGCATGGATCTCTTGTATCGGTT
CTTTTCTCCCGCTCTCTCGCAATAACAATGAACACTGGGTCAATCATAGCCTACACAGGTGAAC
AGAGTAGCGTTTATACAGGGTTTATACGGTGATTCCTACGGCAAAAATTTTTCATTTCTAAAAA
AAAAAAGAAAAATTTTTTGCTTCTGCTGGATAATTTTCAGAGGCAACAAGGAAAAATTAGATGG
CAAAAAGTCGTCTTTCAAGGAAAAATCCCCACCATCTTTCGAGATCCCCTGTAACTTATTGGCA
ACTGAAAGAATGAAAAGGAGGAAAATACAAAATATACTAGAACTGAAAAAAAAAAAGTATAAAT
AGAGAAGATATATGCCAATACTTCACAATGTTCGAATCTATTCTTCATTTGCAGCTATTGTAAA
ATAATAAAACATCAAGAACAAACAAGCTCAACTTGTCTTTTCTAAGAACAAAGAATAAACACAA
AAACAAAAAGTTTTTTTAATTTTAATCAAAAA
SEQ ID NO: 27 > EPK27
TTCAGGCGGCTACTTGTATGTAGCATCCACGTTCATGTTTTGTGGATCAGATTAATGGTATGGA
TATGCACGGTTGAAATGAATCGGCCGACGCTCGGTAGTCGGAAAGAGCCGGGACCGGCCGGCGA
GCATAAACCGGACGCAGTAGGATGTCCTGCACGGGTCTTTTTGTGGGGTGTGGAGAGGGGGGTG
CTTGGAGATGGAAGCCGGTAGAACCGGGCTGCTTGGGGGGATTTGGGGCCGCTGGGCTCCAAAG
AGGGGTAGGCATTTCGTTGGGGTTACGTAATCCGCACCGAAGTGACAACATGGACAATGTGACA
CGTAGATACACGCAGGAAGCAGCTGTCCACACACATTTATCCCGAAAAATAGCCCGCATCACAT
GCACGACTCGTAAAAAGAAAAGAGCTGCGGGCCAAAGGACCAATAAGTGCCGAGGAATGTTAAG
CCAAAAGAACAACGACGATCGCCAGACAGGTTTAGTGGGAGCAGCAGCAGCAGAGGCCGTGCAA
CGGCAGGAGAGAGAGGTCTGGCGAAAAGGAGGAGACGGGCCATGCAGTCGGATTTGCCGTCACG
GGACCGCAACATGCTTTTCATTGCAGTCCTTCAACTATCCATCTCACCTCCCCCAATGGCTTTT
AACTTTCGAATGACGAAAGCACCCCCCTTTGTACAGATGACTATTTGGGACCAATCCAATAGCG
CAATTGGGTTTGCATCATGTATAAAAGGAGCAATCCCCCACTAGTTATAAAGTCACAAGTATCT
CAGTATACCCGTCTAACCACACATTTATCACC
SEQ ID NO: 28 > EPK28
ATCACGCTACACTTAGCTACAGAATAAAGCTCGGTAGCGCCAACAGCGTTGACAAATAGCTCAA
GGGCGTGGAGCACAGGGTTTAGGAGGTTTTAATGGGCGAGAAGGCGCGTAGATGTAGTCTTCCT
CGGTCCCATCGGTAATCACGTGTGTGCCGATTTGCAAGACGAAAAGCCACGAGAATGGGGGGGG
AGAGGGGATGGAAGTCCCCGAACAGCAACCAGCCCTTGCCCTCGTGGACATAACCTTTCACTTG
CCAGAACTCTAAGCGTCACCACGGTATACAAGCGCACGTAGAAGATTGTGGAAGTTGAGCTCGT
GTTGGAATTTCAGTATAAGCGATTCCGTCAGACTCACTGCATCTGGGTCAGAGGGAGGCTTTAG
GACAGGAGCCATCTGTACAGAGGCTACGGAGTGTGGTGGCGGGTTCATGGGTGGCTCAAGCGGT
CTAAAAGCAAAGGTGCGCGGCCGTACGTTATTGTTTGTGTGGGTACGCGATAAATAAAAAATCA
GACAATCGGCTATGGGGGTGACATAAGCGATGAGCAAACTCTATAGCCTCGGCAAACCTGGGCG
TGCACACACTCTTTTCTTCTAACCAAGGGGGTGGTTTAGTTTAGTAGAACCTCGTGAAACTTAC
ATTTACATATATATAAACTTGCATAAATTGGTCAATGCAAGAAATACATATTTGGTCTTTTCTA
ATTCGTAGTTTTTCAAGTTCTTAGATGCTTTCTTTTTCTCTTTTTTACAGATCATCAAGGAAGT
AATTATCTACTTTTTACAACAAATATAAAACA
SEQ ID NO: 29 > EPK29
GTAGGTTGGGTTGGGTGGGAGCACCCCTCCACAGAGTAGAGTCAAACAGCAGCAGCAACATGAT
AGTTGGGGGTGTGCGTGTTAAAGGAAAAAAAAAGAAGCTTGGGTTATATTCCCGCTCTATTTAG
AGGTTGCGGGATAGACGCCGACGGAGGGCAATGGCGCCATGGAACCTTGCGGATATGGGGACGC
CGCGGCGGACTGCGTCCGAACCAGCTCCAGCAGCGTTTTTTCCGGGCCATTGAGCCGACTGCGA
CCCCGCCAACGTGTCTTGGCCCACGCACTCATGTCATGTTGGTGCGGCTTGCACGGGATAATCA
GCAGTCTTTTTCGGAATTTGAAAATGTTTGGACGGACTACGAAAATAAGGAGTTTGATATTAAT
GAGAGTAAGTTTTTCAATGCAATGGAAGAGAAGAACGAAGCTGCAGAAGAGCAAATAGACGCTT
TGAAACAGCAGATCTTGAACATGGATGCATACACCAAACAGCTCTGGGAGCAAGCAAAGTCGTC
CTGGAAGCTGGCTCCTAAAATGGAAATGCATGTGGCAAAGTAATGGCTCCTAGGGTGGACTTCA
AGGATACACTCACATTGATTGGTTGACAAGGTTACGTAAACTTGTCTCCGAAGACGAATTTGTT
TACGAAGATAGCATTTATTTGTAGGTTATTGATTAGGTAAGATTTATTCAACGCAAACTGGGTG
ATGAATATAAATAAGCTCGGGTATTCCTCCGCAACCAGAACTTGAATTATATACGTTTCGATTG
ATTTCCATCAAATTATAAATTCAACAATTGCA
SEQ ID NO: 30 > EPK30
CAGACAGTGACGAGTCATACATTCTCCGTATAATATCGTGTATGTCCAGACGATAGTCGTACTC
GTACTCGTTACTGTAACTACTGTGCGAGTACTCGTGCATGTATCGTAGGTATTGTATGTTCGAG
TACATACACATACGATACCAAACACTGCCCACTGTTCTGTCATGTTAGATCATGGCGGGGCCAC
GTGACTTGCATGCAGGTTTGGCATTGAATATTCAGCGTGGCTACTACAAGTAGTACATACTGTA
TCAATACGATTGTACATACGGTACTGGGTTTCGCAAACAACATGGTAAAATTACAAATGTGACT
ATTTTTCCACTTTTTATTTTGGTACACTGGCCTTTCTTTTTGCCATCAGCGCAATTCCCGACAC
CCGTGCACCGCGACACCCAAAAGTTGTCATTGAAATTTTCTCGCCGTTTGAGATCGCTTTTGGA
AAAAAAGAAAAAAATTACCTGGAAGCTCGTAGAATCCAGGGAGCCGAGGAATAAACTGGGGGTG
CACAGCCTTACACCTTGTTATATATCTCGAGCACACGATCAAAGTGCTACAAAACCAGTACGAC
CACATCAGCGCACAGCATATGCAAAATTTGGACCAGTATCTAAAGAAAATGGCCCGCTGGAGGG
AAATTGTATAAAGATAGACAAATCGACTAAATTTCTAAAACACAAAATAATTTTTTATGATCCA
CATGGATGGAAGTGCTCAAGTTATTGCAATCCTTGGGGGATGAGAACAAGAAGGACTTGATGCG
ACGCAGTGTCGAGGTCTTTACTTGTACCATCT
SEQ ID NO: 31 > EPK31
GTAGGTTGGGTTGGGTGGGAGCACCCCTCCACAGAGTAGAGTCAAACAGCAGCAGCAACATGAT
AGTTGGGGGTGTGCGTGTTAAAGGAAAAAAAAAGAAGCTTGGGTTATATTCCCGCTCTATTTAG
AGGTTGCGGGATAGACGCCGACGGAGGGCAATGGCGCCATGGAACCTTGCGGATATGGGGACGC
CGCGGCGGACTGCGTCCGAACCAGCTCCAGCAGCGTTTTTTCCGGGCCATTGAGCCGACTGCGA
CCCCGCCAACGTGTCTTGGCGCAACATTCGGGAGCAGCTGGAGCGTATGTGTGAGCGGATGGGG
CTTTTGGACGCAAACCAGCCGGTTTTGGACCACGACCGACTGCTTGTGAACGTGCTGAAGAGCA
TTGTGGCTGGCTTTTTCGTCAATGCTGCGCAGCTGAGCCGGTCCGGCGACTCGTACCGGTCGAT
GAAAAAGAACCAGGCGGTGTGGATGCATCCGTCGTCGGTGCTGTTCGGCGTGAAGCCGCCGCCG
AAGCTGGTGATTTGGATGATTATGCATTGTCTCCACATTGTATGCTTCCAAGATTCTGGTGGGA
ATACTGCTGATAGCCTAACGTTCATGATCAAAATTTAACTGTTCTAACCCCTACTTGACAGCAA
TATATAAACAGAAGGAAGCTGCCCTGTCTTAAACCTTTTTTTTATCATCATTATTAGCTTACTT
TCATAATTGCGACTGGTTCCAATTGACAAGCTTTTGATTTTAACGACTTTTAACGACAACTTGA
GAAGATCAAAAAACAACTAATTATTCGAAACG
SEQ ID NO: 32 > EPK32
CAGACAGTGACGAGTCATACATTCTCCGTATAATATCGTGTATGTCCAGACGATAGTCGTACTC
GTACTCGTTACTGTAACTACTGTGCGAGTACTCGTGCATGTATCGTAGGTATTGTATGTTCGAG
TACATACACATACGATACCAAACACTGCCCACTGTTCTGTCATGTTAGATCATGGCGGGGCCAC
GTGACTTGCATGCAGGTTTGGCATTGAATATTCAGCGTGGCTACTACAAGTAGTACATACTGTA
TCAATACGATTGTACATACGGTACTCACCCTTTGCTACAGTATGTACATACAAGGGCGCAACAG
AGCCGAGTTTAATGGTTTGAAACCTAGGTGGAAGAGGGGGGGGCGAGGTATCGTACTGTGGGTG
CGATAGTTCACCAGTCACGCTGGTGGGCCATTCTCTTAGCACATTTCCCCCCTCCCAAGTCCCC
TCAACCCCCAATGTAACCCTCAACCTCCCACAGCTCAGTAGCACACGTGCAACTAGTTAGTAAC
AACCCCCCTCCGTCCAGCTTCTCTTTCACACTGCTTAGAGTTCGACTTCTACTTGAGTGTGGGG
AGATTGCTCACAAGTCTGGACTGCCACGGATGACTAAAGTTTGAGCGTTTAATCCGCCAAATGA
CCACACTGTGCATAAACTCGATTGCCAGCGAAATAGAGTTGCTTTACTAAGCACAAAGTCTGTT
GAGTTGGCTGAGACTTGGATTTATAAAACGCTGCAGCGTCCCTCTCCAGACCTTTTCTGCAACT
TGACATTTTCTTGTTAACGACACCATCACACA
SEQ ID NO: 33 > EPK33
GGGACAAAGAAGAATCTTCGTTCTTCTTTCTTGTTCTCAACTTCCCAGCTTCCGTGTGATTACC
CTCCGGGACAACAGAAAAACTGGCATTCGGTATCCCGGGAATCTGCTGAGAAGGAAAGAAAACG
AAAAAAAAATTGTACATTTGTGTCACATTATGAATTACAGGAAGTCAGAAAACAGGGGGGACAT
GTCTCGCACATGCATGTCCATCAGACGAGACATTATGAGACATGCACGCGTGTGAGACTTAGCT
ACAGAATAAAGCTCGGTAGCGCCAACAGCGTTGACAAATAGCTCAAGGGCGTGGAGCACAGGGT
TTAGGAGGTTTTAATGGGCGAGAAGGCGCGTAGATGTAGTCTTCCTCGGTCCCATCGGTAATCA
CGTGTGTGCCGATTTGCAAGACGAAAAGCCACGAGAATAAACCGGGAGAGGGGATGGAAGTCCC
CGAACAGCAACCAGCCCTTGCCCTCGTGGACATAACCTTTCACTTGCCAGAACTCTAAGCGTCA
CCACGGTATACAAGCGCACGTAGAAGATCCTCGAATTTGAGACGAGTCACGGCCCCATTCGCCC
GCGCAATGGCTCGCCAACGCCCGGTCTTTTGCACCACATCAGGTTACCCCAAGCCAAACCTTTG
TGTTAAAAAGCTTAACATATTATACCGAACGTAGGTTTGGGCGGGCTTGCTCCGTCTGTCCAAG
GCAACATTTATATAAGGGTCTGCATCGCCGGCTCAATTGAATCTTTTTTCTTCTTCTCTTCTCT
ATATTCATTCTTGAATTAAACACACATCAACA

1. An artificial yeast promoter region, comprising a TATA box at position −90±15 nucleotides (nt) and/or a TATA box at position −160±15 nt, an enhancer element at position −350±25 nucleotides, and a second enhancer element at position −600±50 nucleotides,
wherein said enhancer element at position −350±25 nucleotides is selected from a AZF1 binding element and a MSN4 binding element,
wherein said enhancer element at position −600±50 nucleotides is selected from a GCR1 binding element, a GCR2 binding element, and a PHD1 binding element,
wherein all positions are relative to the start codon ATG, and
wherein the sequences in between the indicated TATA boxes and enhancer elements lack known repressor elements.

2. The artificial yeast promoter region according to claim 1, wherein the enhancer element at position −350±25 nucleotides is selected from 5′-AAMRGMA and 5′-RVCCCCYR.

3. The artificial yeast promoter region according to claim 1, wherein the second enhancer element at position −600±50 nucleotides is selected from 5′-WGGAWGMY, 5′-WGGAAGNM, and 5′-VMTGCRKV.

4. The artificial yeast promoter region according to claim 1, wherein the promoter region is able to drive expression of a downstream protein in at least one of Kluyveromyces marxianus, Kluyveromyces lactis, Komagataella pastoris, Komagataella phaffii, Ogataea angusta, Yarrowia lipolytica, Schizosaccharomyces pombe, Rhodotorula mucilaginosa, Candida famata, and Saccharomyces cerevisiae.

5. The artificial yeast promoter region according to claim 1, wherein the nucleotide sequences of the promoter region are identical to any one of SEQ ID NOs: 1-33.

6. An expression construct comprising the artificial yeast promoter region of claim 1, for expression of a protein of interest in a yeast.

7. The expression construct according to claim 6, comprising a nucleotide sequence encoding a protein of interest under control of the artificial yeast promoter region of claim 1.

8. The expression construct according to claim 7, wherein the nucleotide sequence encoding the protein of interest is codon optimized for expression in a yeast host cell.

9. A yeast host cell, comprising the artificial yeast promoter region of claim 1.

10. A method of producing a protein of interest in a yeast host cell, comprising
providing an expression construct according to claim 7;
transforming a yeast cell with the expression construct; and
expressing the protein of interest.

11. A method of producing a protein of interest in a yeast host cell, comprising
providing the yeast host cell comprising the expression construct according to claim 9; and
expressing the protein of interest.

12. The method according to claim 10, wherein the protein of interest is tagged.

13. The method according to claim 10, wherein the protein of interest is co-expressed in the yeast cell with one or more of a protein disulfide isomerase, a flavin-linked sulfhydryl oxidase, and an oxidoreductase.

14. A method according to claim 10, wherein the protein of interest is part of a metabolic pathway, the method further comprising
modulating the expression levels of one or more other enzymes in the metabolic pathway.

15. The method of claim 14, wherein said metabolic pathway is selected from the production of a biofuel, the breakdown of a carbohydrate, the production of a biopolyester, the production of a tocochromanol, and the production of an alkaloid.

16. The artificial yeast promoter region of claim 4, wherein the organism is Komagataella phaffii.

17. The artificial yeast promoter region of claim 4, wherein the organism is Yarrowia lipolytica.

18. The artificial yeast promoter region of claim 4, wherein the organism is Saccharomyces cerevisiae.

19. The method of claim 10, further comprising the step of at least partially purifying the protein of interest.

20. The method of claim 11, further comprising the step of at least partially purifying the protein of interest.
